SEQUENCE LISTING (87 sequences)

SEQ ID NO: 1 - protein, 9 aa, Artificial Sequence
  Synthetic construct
ESVDNYGIS

SEQ ID NO: 2 - protein, 3 aa, Artificial Sequence
  Synthetic construct
AAS

SEQ ID NO: 3 - protein, 9 aa, Artificial Sequence
  Synthetic construct
QQSKEVPWT

SEQ ID NO: 4 - protein, 8 aa, Artificial Sequence
  Synthetic construct
GDSITSGY

SEQ ID NO: 5 - protein, 7 aa, Artificial Sequence
  Synthetic construct
ISYSGST

SEQ ID NO: 6 - protein, 12 aa, Artificial Sequence
  Synthetic construct
ASPNYPFYAMDY

SEQ ID NO: 7 - protein, 134 aa, Artificial Sequence
  Synthetic construct
METDTLLLWVLLLWVPGSTGEIVLTQSPATLSLSPGERATLSCRASESVDNYGISFLNWFQQKPGQAPRLLIYAASNRATGIPARFSGSGSGTDFTLTISSLEPEDFAVYYCQQSKEVPWTFGGGTKVEIKRTV

SEQ ID NO: 8 - protein, 134 aa, Artificial Sequence
  Synthetic construct
METDTLLLWVLLLWVPGSTGDIVLTQSPATLSLSPGERATLSCRASESVDNYGISFMNWFQQKPGQAPRLLIYAASNRATGIPARFSGSGSGTDFTLTISSLEPEDFAVYYCQQSKEVPWTFGGGTKVEIKRTV

SEQ ID NO: 9 - protein, 134 aa, Artificial Sequence
  Synthetic construct
METDTLLLWVLLLWVPGSTGDIVMTQSPSSLSASVGDRVTITCRASESVDNYGISFMNWFQQKPGKAPKLLIYAASNLGSGVPSRFSGSGSGTDFTLTISSLQPEDFATYYCQQSKEVPWTFGQGTKVEIKRTV

SEQ ID NO: 10 - protein, 142 aa, Artificial Sequence
  Synthetic construct
MDPKGSLSWRILLFLSLAFELSYGQVQLQESGPGLVKPSQTLSLTCTVSGDSITSGYWNWIRQHPGKGLEYIGYISYSGSTYYNPSLKSRVTISRDTSKNQFSLKLSSVTAADTAVYYCASPNYPFYAMDYWGQGTLVTVSS

SEQ ID NO: 11 - protein, 142 aa, Artificial Sequence
  Synthetic construct
MDPKGSLSWRILLFLSLAFELSYGEVQLQESGPGLVKPSQTLSLTCTVSGDSITSGYWNWIRQHPGKGLEYIGYISYSGSTYYNPSLKSRVTISRDTSKNQYSLKLSSVTAADTAVYYCASPNYPFYAMDYWGQGTLVTVSS

SEQ ID NO: 12 - protein, 142 aa, Artificial Sequence
  Synthetic construct
MDPKGSLSWRILLFLSLAFELSYGEVQLQESGPGLVKPSETLSLTCSVSGDSITSGYWNWIRQPPGKGLEYIGYISYSGSTYYNPSLKSRVTISRDTSKNQYSLRLSSVTAADTALYYCASPNYPFYAMDYWGQGTRVTVSS

SEQ ID NO: 13 - protein, 46 aa, Artificial Sequence
  Synthetic construct
METDTLLLWVLLLWVPGSTGEIVLTQSPATLSLSPGERATLSCRAS

SEQ ID NO: 14 - protein, 18 aa, Artificial Sequence
  Synthetic construct
FLNWFQQKPGQAPRLLIY

SEQ ID NO: 15 - protein, 36 aa, Artificial Sequence [sequence omitted]

SEQ ID NO: 16 - protein, 13 aa, Artificial Sequence
  Synthetic construct
FGGGTKVEIKRTV

SEQ ID NO: 17 - protein, 46 aa, Artificial Sequence
  Synthetic construct
METDTLLLWVLLLWVPGSTGDIVLTQSPATLSLSPGERATLSCRAS

SEQ ID NO: 18 - protein, 18 aa, Artificial Sequence
  Synthetic construct
FMNWFQQKPGQAPRLLIY

SEQ ID NO: 19 - protein, 36 aa, Artificial Sequence [sequence omitted]

SEQ ID NO: 20 - protein, 13 aa, Artificial Sequence
  Synthetic construct
FGGGTKVEIKRTV

SEQ ID NO: 21 - protein, 46 aa, Artificial Sequence
  Synthetic construct
METDTLLLWVLLLWVPGSTGDIVMTQSPSSLSASVGDRVTITCRAS

SEQ ID NO: 22 - protein, 18 aa, Artificial Sequence
  Synthetic construct
FMNWFQQKPGKAPKLLIY

SEQ ID NO: 23 - protein, 36 aa, Artificial Sequence
  Synthetic construct
NLGSGVPSRFSGSGSGTDFTLTISSLQPEDFATYYC

SEQ ID NO: 24 - protein, 13 aa, Artificial Sequence
  Synthetic construct
FGQGTKVEIKRTV

SEQ ID NO: 25 - protein, 49 aa, Artificial Sequence
  Synthetic construct
MDPKGSLSWRILLFLSLAFELSYGQVQLQESGPGLVKPSQTLSLTCTVS

SEQ ID NO: 26 - protein, 17 aa, Artificial Sequence
  Synthetic construct
WNWIRQHPGKGLEYIGY

SEQ ID NO: 27 - protein, 38 aa, Artificial Sequence
  Synthetic construct
YYNPSLKSRVTISRDTSKNQFSLKLSSVTAADTAVYYC

SEQ ID NO: 28 - protein, 11 aa, Artificial Sequence
  Synthetic construct
WGQGTLVTVSS

SEQ ID NO: 29 - protein, 49 aa, Artificial Sequence
  Synthetic construct
MDPKGSLSWRILLFLSLAFELSYGEVQLQESGPGLVKPSQTLSLTCTVS

SEQ ID NO: 30 - protein, 17 aa, Artificial Sequence
  Synthetic construct
WNWIRQHPGKGLEYIGY

SEQ ID NO: 31 - protein, 38 aa, Artificial Sequence
  Synthetic construct
YYNPSLKSRVTISRDTSKNQYSLKLSSVTAADTAVYYC

SEQ ID NO: 32 - protein, 11 aa, Artificial Sequence
  Synthetic construct
WGQGTLVTVSS

SEQ ID NO: 33 - protein, 49 aa, Artificial Sequence
  Synthetic construct
MDPKGSLSWRILLFLSLAFELSYGEVQLQESGPGLVKPSETLSLTCSVS

SEQ ID NO: 34 - protein, 17 aa, Artificial Sequence
  Synthetic construct
WNWIRQPPGKGLEYIGY

SEQ ID NO: 35 - protein, 38 aa, Artificial Sequence
  Synthetic construct
YYNPSLKSRVTISRDTSKNQYSLRLSSVTAADTALYYC

SEQ ID NO: 36 - protein, 11 aa, Artificial Sequence
  Synthetic construct
WGQGTRVTVSS

SEQ ID NO: 37 - protein, 12 aa, Artificial Sequence
  Synthetic construct
GGGSGGGSGGGS

SEQ ID NO: 38 - protein, 6 aa, Artificial Sequence
  Synthetic construct
QDISNY

SEQ ID NO: 39 - protein, 3 aa, Artificial Sequence
  Synthetic construct
YTS

SEQ ID NO: 40 - protein, 9 aa, Artificial Sequence
  Synthetic construct
HQFSELPWT

SEQ ID NO: 41 - protein, 10 aa, Artificial Sequence
  Synthetic construct
GFSLSTSGMG

SEQ ID NO: 42 - protein, 7 aa, Artificial Sequence
  Synthetic construct
IWWDDDK

SEQ ID NO: 43 - protein, 10 aa, Artificial Sequence
  Synthetic construct
ARSFGYGLDY

SEQ ID NO: 44 - protein, 130 aa, Artificial Sequence
  Synthetic construct
METDTLLLWVLLLWVPGSTGDIQMTQSPSSLSASVGDRVTITCQASQDISNYLNWYQQKPGKAPKLLIYYTSSLHTGVPSRFSGSGSGTDYTFTISSLQPEDIATYYCHQFSELPWTFGGGTKVEIKRTV

SEQ ID NO: 45 - protein, 130 aa, Artificial Sequence
  Synthetic construct
METDTLLLWVLLLWVPGSTGDIQMTQSPSSLSASVGDRVTITCSASQDISNYLNWYQQKPGKAPKLLIYYTSSLHTGVPSRFSGSGSGTDYTLTISSLQPEDIATYYCHQFSELPWTFGGGTKVEIKRTV

SEQ ID NO: 46 - protein, 130 aa, Artificial Sequence
  Synthetic construct
METDTLLLWVLLLWVPGSTGDIQMTQSPSSLSASVGDRVTITCQASQDISNYLNWYQQKPGKTPKLLIYYTSSLHTGVPSRFSGSGSGTDYTLTISSLQPEDIATYYCHQFSELPWTFGGGTKVEIKRTV

SEQ ID NO: 47 - protein, 142 aa, Artificial Sequence
  Synthetic construct
MDPKGSLSWRILLFLSLAFELSYGQVTLKESGPTLVKPTQTLTLTCTFSGFSLSTSGMGVGWIRQPPGKALEWLAHIWWDDDKYYNPSLKSRLTITKDTSKNQVVLTMTNMDPVDTATYYCARSFGYGLDYWGQGTLVTVSS

SEQ ID NO: 48 - protein, 142 aa, Artificial Sequence
  Synthetic construct
MDPKGSLSWRILLFLSLAFELSYGQVTLKESGPTLVKPTQTLTLTCTFSGFSLSTSGMGVGWIRQPPGKALEWLAHIWWDDDKYYNSSLKSRLTITKDTSKNQVVLTMTNMDPVDTATYYCARSFGYGLDYWGQGTLVTVSS

SEQ ID NO: 49 - protein, 142 aa, Artificial Sequence
  Synthetic construct
MDPKGSLSWRILLFLSLAFELSYGQVTLKESGPALVKPTQTLTLTCTFSGFSLSTSGMGVGWIRQPPGKALEWLAHIWWDDDKYYNTSLKSRLTITKDTSKNQVVLKMTNMDPVDTATYYCARSFGYGLDYWGQGTLVTVSS

SEQ ID NO: 50 - protein, 46 aa, Artificial Sequence
  Synthetic construct
METDTLLLWVLLLWVPGSTGDIQMTQSPSSLSASVGDRVTITCQAS

SEQ ID NO: 51 - protein, 17 aa, Artificial Sequence
  Synthetic construct
LNWYQQKPGKAPKLLIY

SEQ ID NO: 52 - protein, 36 aa, Artificial Sequence
  Synthetic construct
SLHTGVPSRFSGSGSGTDYTFTISSLQPEDIATYYC

SEQ ID NO: 53 - protein, 13 aa, Artificial Sequence
  Synthetic construct
FGGGTKVEIKRTV

SEQ ID NO: 54 - protein, 46 aa, Artificial Sequence
  Synthetic construct
METDTLLLWVLLLWVPGSTGDIQMTQSPSSLSASVGDRVTITCSAS

SEQ ID NO: 55 - protein, 17 aa, Artificial Sequence
  Synthetic construct
LNWYQQKPGKAPKLLIY

SEQ ID NO: 56 - protein, 36 aa, Artificial Sequence
  Synthetic construct
SLHTGVPSRFSGSGSGTDYTLTISSLQPEDIATYYC

SEQ ID NO: 57 - protein, 13 aa, Artificial Sequence
  Synthetic construct
FGGGTKVEIKRTV

SEQ ID NO: 58 - protein, 46 aa, Artificial Sequence; identical to SEQ ID NO: 50
  Synthetic construct

SEQ ID NO: 59 - protein, 17 aa, Artificial Sequence
  Synthetic construct
LNWYQQKPGKTPKLLIY

SEQ ID NO: 60 - protein, 36 aa, Artificial Sequence; identical to SEQ ID NO: 56
  Synthetic construct

SEQ ID NO: 61 - protein, 13 aa, Artificial Sequence
  Synthetic construct
FGGGTKVEIKRTV

SEQ ID NO: 62 - protein, 49 aa, Artificial Sequence
  Synthetic construct
MDPKGSLSWRILLFLSLAFELSYGQVTLKESGPTLVKPTQTLTLTCTFS

SEQ ID NO: 63 - protein, 17 aa, Artificial Sequence
  Synthetic construct
VGWIRQPPGKALEWLAH

SEQ ID NO: 64 - protein, 38 aa, Artificial Sequence
  Synthetic construct
YYNPSLKSRLTITKDTSKNQVVLTMTNMDPVDTATYYC

SEQ ID NO: 65 - protein, 11 aa, Artificial Sequence
  Synthetic construct
WGQGTLVTVSS

SEQ ID NO: 66 - protein, 49 aa, Artificial Sequence; identical to SEQ ID NO: 62
  Synthetic construct

SEQ ID NO: 67 - protein, 17 aa, Artificial Sequence
  Synthetic construct
VGWIRQPPGKALEWLAH

SEQ ID NO: 68 - protein, 38 aa, Artificial Sequence
  Synthetic construct
YYNSSLKSRLTITKDTSKNQVVLTMTNMDPVDTATYYC

SEQ ID NO: 69 - protein, 11 aa, Artificial Sequence
  Synthetic construct
WGQGTLVTVSS

SEQ ID NO: 70 - protein, 49 aa, Artificial Sequence
  Synthetic construct
MDPKGSLSWRILLFLSLAFELSYGQVTLKESGPALVKPTQTLTLTCTFS

SEQ ID NO: 71 - protein, 17 aa, Artificial Sequence
  Synthetic construct
VGWIRQPPGKALEWLAH

SEQ ID NO: 72 - protein, 38 aa, Artificial Sequence
  Synthetic construct
YYNTSLKSRLTITKDTSKNQVVLKMTNMDPVDTATYYC

SEQ ID NO: 73 - protein, 11 aa, Artificial Sequence
  Synthetic construct
WGQGTLVTVSS

SEQ ID NO: 74 - protein, 9 aa, Artificial Sequence
  Synthetic construct
TGAVTSGNY

SEQ ID NO: 75 - protein, 3 aa, Artificial Sequence
  Synthetic construct
GTK

SEQ ID NO: 76 - protein, 9 aa, Artificial Sequence
  Synthetic construct
VLWYSNRWV

SEQ ID NO: 77 - protein, 8 aa, Artificial Sequence
  Synthetic construct
GFTFNKYA

SEQ ID NO: 78 - protein, 10 aa, Artificial Sequence
  Synthetic construct
IRSKYNNYAT

SEQ ID NO: 79 - protein, 16 aa, Artificial Sequence
  Synthetic construct
VRHGNFGNSYISYWAY

SEQ ID NO: 80 - protein, 249 aa, Artificial Sequence
  Synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 5 aa, Artificial Sequence
  Synthetic construct
SSVSY

SEQ ID NO: 82 - protein, 3 aa, Artificial Sequence
  Synthetic construct
DTS

SEQ ID NO: 83 - protein, 9 aa, Artificial Sequence
  Synthetic construct
QQWSSNPFT

SEQ ID NO: 84 - protein, 8 aa, Artificial Sequence
  Synthetic construct
GYTFTRYT

SEQ ID NO: 85 - protein, 8 aa, Artificial Sequence
  Synthetic construct
INPSRGYT

SEQ ID NO: 86 - protein, 12 aa, Artificial Sequence
  Synthetic construct
ARYYDDHYCLDY

SEQ ID NO: 87 - protein, 241 aa, Artificial Sequence
  Synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

(19) United States
(12) Patent Application Publication
(10) Pub. No.: US 2020/0199232 A1
(43) Pub. Date: Jun. 25, 2020

(54) BAFF-R BISPECIFIC T-CELL ENGAGER ANTIBODY

(71) Applicant: City of Hope, Duarte, CA (US)

(72) Inventors: Hong Qin, Upland, CA (US); Larry W. Kwak, Pasadena, CA (US)

(21) Appl. No.: 16/721,485

(22) Filed: Dec. 19, 2019

Related U.S. Application Data
(60) Provisional application No. 62/782,317, filed on Dec. 19, 2018.

(51) Int. Cl. C07K 16/28; A61P 35/00

(52) U.S. Cl. CPC C07K 16/2809; C07K 16/2878; A61P 35/00; C07K 2317/565; C07K 2317/567; A61K 2039/505; C07K 2317/622; C07K 2317/92; C07K 2317/31; C07K 2317/73; C07K 2317/55

(57) ABSTRACT

Provided herein are recombinant proteins (e.g., a bispecific antibody) capable of binding BAFF-R. The recombinant proteins (e.g., bispecific antibody) provided herein are, inter alia, useful for the treatment of cancer and autoimmune disease.

### CROSS-REFERENCES TO RELATED APPLICATIONS

This application claims the benefit of U.S. Provisional Application No. 62/782,317, filed Dec. 19, 2018, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-715001WO_SEQUENCE_LISTING_ST25, created Dec. 12, 2019, 45,056 bytes, machine format IBM-PC, MS Windows operating system, is hereby incorporated by reference BACKGROUND

Antibody therapy is one of the most successful immunotherapies available in the clinic to treat hematological malignancies. An exemplary case is rituximab, which targets CD20 and elicits a cytotoxic effect against B cell lymphomas. However, a major concern regarding rituximab is the emergence of rituximab-resistance thought to be due to the down-regulation of CD20, thus hindering antibodies from binding the target cell. Therefore, there is a need in the art for new therapeutic antibodies against an alternative, specific target on malignant cells. Provided herein are compositions of BAFF-R bispecific antibodies and methods of using the same addressing these and other problems in the art.

### BRIEF SUMMARY

In one aspect, a recombinant protein including: (i) a first antibody region capable of binding an effector cell ligand; and (ii) a second antibody region including: (a) a light chain variable domain and a heavy chain variable domain is provided. The light chain variable domain includes a CDR L1 as set forth in SEQ ID NO: 1, a CDR L2 as set forth in SEQ ID NO:2 and a CDR L3 as set forth in SEQ ID NO:3. The heavy chain variable domain includes a CDR H1 as set forth in SEQ ID NO:4, a CDR H2 as set forth in SEQ ID NO:5, and a CDR H3 as set forth in SEQ ID NO:6.

In one aspect, a recombinant protein including: (i) a first antibody region capable of binding an effector cell ligand; and (ii) a second antibody region including: (a) a light chain variable domain and a heavy chain variable domain is provided. The light chain variable domain includes a CDR L1 as set forth in SEQ ID NO:38, a CDR L2 as set forth in SEQ ID NO:39 and a CDR L3 as set forth in SEQ ID NO:40. The heavy chain variable domain includes a CDR H1 as set forth in SEQ ID NO:41, a CDR H2 as set forth in SEQ ID NO:42, and a CDR H3 as set forth in SEQ ID NO:43.

In an aspect is provided a pharmaceutical composition including a therapeutically effective amount of a recombinant protein as provided herein including embodiments thereof and a pharmaceutically acceptable excipient.

In an aspect is provided a method of treating cancer in a subject in need thereof, the method including administering to a subject a therapeutically effective amount of a recombinant protein as described herein, including embodiments thereof, thereby treating cancer in the subject.

In another aspect is provided a method of treating an autoimmune disease in a subject in need thereof, the method including administering to a subject a therapeutically effective amount of a recombinant protein as described herein, including embodiments thereof, thereby treating an autoimmune disease in the subject.

### BRIEF DESCRIPTION OF THE DRAWINGS

FIG. 1A-1B. Generation of BAFF-R bispecific antibody (BAFF-R BiTE, recombinant protein provided herein including embodiments thereof) by knob-hole technology. FIG. 1A. Schematic of BAFF-R BiTE. FIG. 1B. Reducing and non-reducing gel condition for BAFF-R BiTE (H90Bi). Controls included control/irrelevant BiTE (Cont. Bi, 4-4Bi) and a humanized BAFF-R antibody (e.g., H90 Ab also referred to herein as “humanized C90”, and depending on the framework sequences also referred to as “humanized C90-1”, “humanized C90-2”, “humanized C90-3”, “C90”, “C90-1”, “C90-2”, or “C90-3”).

FIG. 2. Binding specificity of bispecific antibodies. FACS histograms of BAFF-R/CD3 bispecific antibody (H90 BiTE) binding wildtype mouse fibroblast L cells, engineered BAFF-R expressing L cells (B2D L cells), and T cells isolated from healthy donors.

FIG. 3. Specific cytotoxicity of bispecific antibodies against BAFF-R-expressing L cells. Plots show calculated specific cytotoxicity percentage from a chromium release assay. BAFF-R/CD3 bispecific antibody (H90 BiTE) was incubated with chromium labeled target wildtype (left) or BAFF-R expressing L cells (right) along with isolated effector CD8 T cells from a single healthy donor. The ratio of effector cells to isolated CD8 T cells was 10:1. Incubation time was 4 hours.

FIG. 4. Cytotoxicity of bispecific antibodies against malignant B-cell lines. Plots show calculated specific cytotoxicity percentage from a chromium release assay against various malignant B-cell lines as shown. Ratio of effector cells to isolated CD8 T cells was 10:1. Incubation time was 4 hours.

FIG. 5. Dose-dependent cytotoxicity of bispecific antibodies. Plots show calculated specific cytotoxicity percentage from a chromium release assay against JeKo-1 mantle cell lymphoma and normal isolated B cells. Ratio of effector cells to isolated CD8 T cells was 10:1. Incubation time was 4 hours.

FIG. 6. In vivo study schedule. Time line of the treatment schedule to challenge NSG mice with tumors and treat with BAFF-R BiTE. Schematic shows treatment schedule of Z-138 MCL tumor model with administration of time points indicated on a horizontal line. Bioluminescent imaging is performed weekly and mice are observed up to 90 days post tumor challenge.

FIG. 7. Z-138 model treated with BAFF-R BiTE. Bioluminescence images of groups of NSG mice (n=5/group) following intravenous (IV) tumor challenge (5×10⁴ cells/mouse) on day 0 with Z-138 MCL. Experimental group (BAFF-R BiTE 300 μg) received 300 μg BAFF-R BiTE+5×10⁶ T cells. Control groups either received 5×10⁶ T cells from the same donor or saline (PBS).

FIG. 8A-8C. FACS plots of BAFF-R BiTE antibody staining showing binding to parental L cells (FIG. 8A) compared with BAFF-R-positive L cells (B2D) (FIG. 8B) or donor T cells (FIG. 8C). Staining included secondary antibody and BAFF-R humanized antibodies as controls for the L cells, and combinations secondary and CD3 antibody as controls for the T cells.

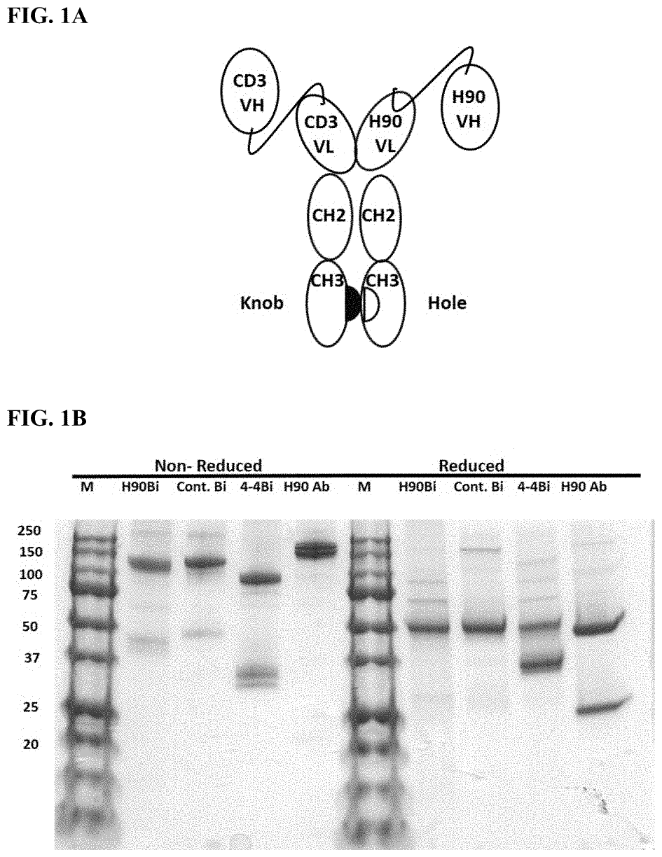

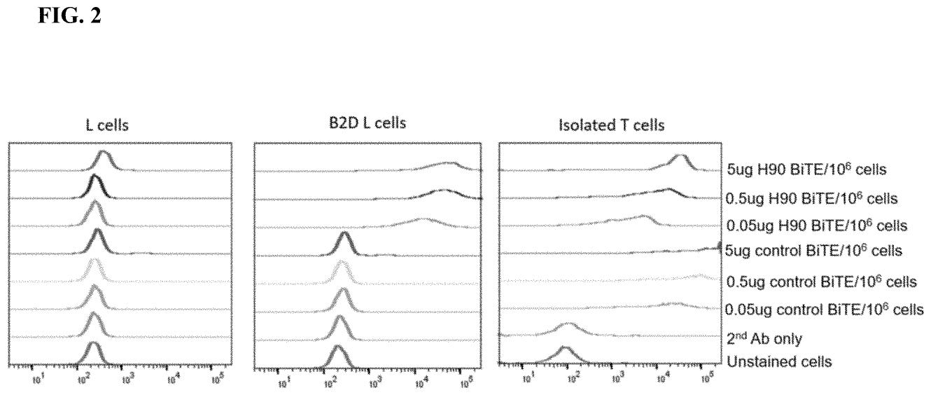

### DETAILED DESCRIPTION

### I. Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length. The present invention includes polypeptides that are substantially identical to any of SEQ ID NOs:7-12 or SEQ ID NOs:44-49.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

An amino acid residue in an antibody “corresponds” to a given residue when it occupies the same essential structural position within the antibody as the given residue. For example, a selected residue in a comparison antibody corresponds to position 48 (according to the Kabat numbering system as described herein) in an antibody provided herein when the selected residue occupies the same essential spatial or structural relationship to Kabat position 48 as assessed using applicable methods in the art. For example, a comparison antibody may be aligned for maximum sequence homology with the antibody provided herein and the position in the aligned comparison antibody that aligns with Kabat position 48 may be determined to correspond to it. Alternatively, instead of (or in addition to) a primary sequence alignment as described above, a three dimensional structural alignment can also be used, e.g., where the structure of the comparison antibody is aligned for maximum correspondence with an antibody provided herein and the overall structures compared. In this case, an amino acid that occupies the same essential position as Kabat position 48 in the structural model may be said to correspond.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety. A “recombinant protein” refers to a protein that can result from the expression of recombinant DNA.

The term “peptidyl” and “peptidyl moiety” means a monovalent peptide.

Antibodies are large, complex molecules (molecular weight of ˜150,000 Da or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site (paratope), which docks onto the target antigen (epitope). The position and length of the CDRs have been precisely defined by Kabat, E. et al., Sequences of Proteins of Immunological Interest, U.S.

Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The term “bispecific T-cell engager (BiTE)”, “BiTe” or “bispecific antibody” as provided herein is used according to its conventional meaning well known in the art and refers to a bispecific recombinant protein capable to bind to two different antigens. For example, simultaneously. In contrast to traditional monoclonal antibodies, BiTE antibodies consist of two independently different antibody regions (e.g., two single-chain variable fragments (scFv)), each of which binds a different antigen. One antibody region may engage effector cells (e.g., T cells) by binding an effector cell-specific antigen (e.g., CD3 molecule) and the second antibody region may bind a target cell (e.g., cancer cell or autoimmune-reactive cell) through a cell surface antigen (e.g., BAFF-R) expressed by said target cell. Binding of the BiTE to the two antigens will link the effector cell (e.g., T cell) to the target cell (e.g., tumor cell) and activate the effector cell (e.g., T cell) via effector cell-specific antigen signaling (e.g., CD3 signaling). The activated effector cell (e.g., T cell) will then exert cytotoxic activity against the target cell (e.g., tumor cells).

The terms “CDR L1,” “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1,” “CDR H2” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3.

The terms “FR L1,” “FR L2,” “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1,” “FR H2,” “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

The term “antibody” is used according to its commonly known meaning in the art.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially a Fab with part of the hinge region (see Fundamental Immunology (Paul ed., 3rd ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) or light chain variable region and variable heavy chain (VH) or heavy chain variable region refer to these light and heavy chain regions, respectively. The terms variable light chain (VL) and light chain variable region as referred to herein may be used interchangeably. The terms variable heavy chain (VH) and heavy chain variable region as referred to herein may be used interchangeably. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (CH, CL, VH, and VL, respectively). The paratope or antigen-binding site is formed on the N-terminus of the antigen binding domain. The two variable domains of an antigen binding domain typically bind the epitope on an antigen.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

An “antibody region” as provided herein refers to a monovalent or multivalent protein moiety that forms part of the protein provided herein including embodiments thereof and that is capable of binding an antigen (epitope). The antibody region provided herein may include a domain of an antibody or fragment (e.g., Fab) thereof. Thus, the antibody region may include a light chain variable domain (VL) and/or a heavy chain variable domain (VH). In embodiments, the antibody region provided herein includes a light chain variable (VL) domain. In embodiments, the antibody region is a light chain variable (VL) domain.

As described above, a “light chain variable (VL) domain” as provided herein refers to the variable region of the light chain of an antibody, an antibody variant or fragment thereof. Likewise, the “heavy chain variable (VH) domain” as provided herein refers to the variable region of the heavy chain of an antibody, an antibody variant or fragment thereof. The light chain variable domain and the heavy chain variable domain together form the paratope, which binds an antigen (epitope). The paratope or antigen-binding site is formed at the N-terminus of an antibody, an antibody variant or fragment thereof. In embodiments, the light chain variable (VL) domain includes CDR L1, CDR L2, CDR L3 and FR L1, FR L2, FR L3 and FR L4 (framework regions) of an antibody light chain. In embodiments, the heavy chain variable (VH) domain includes CDR H1, CDR H2, CDR H3 and FR H1, FR H2, FR H3 and FR H4 (framework regions) of an antibody heavy chain. In embodiments, the light chain variable (VL) domain and a light chain constant (CL) domain form part of an antibody light chain. In embodiments, the heavy chain variable (VH) domain and a heavy chain constant (CH1) domain form part of an antibody heavy chain. In embodiments, the heavy chain variable (VH) domain and one or more heavy chain constant (CH1, CH2, or CH3) domains form part of an antibody heavy chain. Thus, in embodiments, the light chain variable (VL) domain forms part of an antibody. In embodiments, the heavy chain variable (VH) domain forms part of an antibody. In embodiments, the light chain variable (VL) domain forms part of a therapeutic antibody. In embodiments, the heavy chain variable (VH) domain forms part of a therapeutic antibody. In embodiments, the light chain variable (VL) domain forms part of a human antibody. In embodiments, the heavy chain variable (VH) domain forms part of a human antibody. In embodiments, the light chain variable (VL) domain forms part of a humanized antibody. In embodiments, the heavy chain variable (VH) domain forms part of a humanized antibody. In embodiments, the light chain variable (VL) domain forms part of a chimeric antibody. In embodiments, the heavy chain variable (VH) domain forms part of a chimeric antibody. In embodiments, the light chain variable (VL) domain forms part of an antibody fragment. In embodiments, the heavy chain variable (VH) domain forms part of an antibody fragment. In embodiments, the light chain variable (VL) domain forms part of an antibody variant. In embodiments, the heavy chain variable (VH) domain forms part of an antibody variant. In embodiments, the light chain variable (VL) domain forms part of a Fab. In embodiments, the heavy chain variable (VH) domain forms part of a Fab. In embodiments, the light chain variable (VL) domain forms part of a scFv. In embodiments, the heavy chain variable (VH) domain forms part of a scFv. In embodiments, the light chain variable (VL) domain forms part of a bispecific antibody. In embodiments, the heavy chain variable (VH) domain forms part of a bispecific antibody.

The term “isolated,” when applied to a protein, denotes that the protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a protein gives rise to essentially one band in an electrophoretic gel. Particularly, it means that the protein is at least 85% pure, more preferably at least 95% pure, and most preferably at least 99% pure.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Typically a specific or selective reaction will be at least twice background signal or noise and more typically more than 10 to 100 times background.

A “humanized antibody” is a genetically engineered antibody in which at least one CDR (or functional fragment thereof) from a mouse antibody (“donor antibody,” which can also be rat, hamster or other non-human species) are grafted onto a human antibody (“acceptor antibody”). In embodiments, more than one mouse CDR is grafted (e.g., all six mouse CDRs are grafted). The sequence of the acceptor antibody can be, for example, a mature human antibody sequence (or fragment thereof), a consensus sequence of a human antibody sequence (or fragment thereof), or a germline region sequence (or fragment thereof). Thus, a humanized antibody may be an antibody having one or more CDRs from a donor antibody and a variable region framework (FR). The FR may form part of a constant region and/or a variable region within a human antibody. In addition, in order to retain high binding affinity, amino acids in the human acceptor sequence may be replaced by the corresponding amino acids from the donor sequence, for example where: (1) the amino acid is in a CDR or (2) the amino acid is in the human framework region (e.g., the amino acid is immediately adjacent to one of the CDRs). See, U.S. Pat. Nos. 5,530,101 and 5,585,089, incorporated herein by reference, which provide detailed instructions for construction of humanized antibodies. Although humanized antibodies often incorporate all six CDRs (e.g., as defined by Kabat, but often also including hypervariable loop H1 as defined by Chothia) from a mouse antibody, they can also be made with fewer mouse CDRs and/or less than the complete mouse CDR sequence (e.g., a functional fragment of a CDR) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al., Journal of Immunology, 164:1432-1441, 2000).

Typically a humanized antibody as provided herein may include (i) a light chain variable region comprising at least one CDR (often three CDRs) from a mouse antibody (also referred to herein as a mouse CDR) and a human variable region framework; and (ii) a heavy chain variable region comprising at least one CDR (often three CDRs) from the mouse antibody and a human variable region framework (FR). The light and heavy chain variable region frameworks (FRs) may each be a mature human antibody variable region framework sequence (or fragment thereof), a germline variable region framework sequence (combined with a J region sequence) (or fragment thereof), or a consensus sequence of a human antibody variable region framework sequence (or fragment thereof). In embodiments, the humanized antibody includes a light chain variable region as described in (i), a heavy chain variable region as described in (ii) together with a light chain human constant region and a heavy chain human constant region.

Other approaches to design humanized antibodies may also be used to achieve the same result as the methods in U.S. Pat. Nos. 5,530,101 and 5,585,089 described above, for example, “superhumanization” (see, Tan et al., J. Immunol. 169: 1119, 2002, and U.S. Pat. No. 6,881,557) or the method of Studnicak et al., Protein Eng. 7:805, 1994. Moreover, other approaches to produce genetically engineered, reduced-immunogenicity mAbs include “reshaping,” “hyperchimerization” and veneering/resurfacing, as described, e.g., in Vaswami et al., Annals of Allergy, Asthma and Immunology 81:105, 1998; Roguska et al., Protein Eng. 9:895, 1996; and U.S. Pat. Nos. 6,072,035 and 5,639,641.

A “ligand” refers to an agent, e.g., a polypeptide or other molecule, capable of binding to a receptor molecule (e.g., an antibody).

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, an antibody as described herein and a BAFF-R protein. In embodiments contacting includes, for example, allowing a humanized antibody as described herein to interact with BAFF-R.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

As defined herein, the term “inhibition,” “inhibit,” “inhibiting” and the like in reference to a protein-inhibitor interaction means negatively affecting (e.g., decreasing) the activity or function of the protein relative to the activity or function of the protein in the absence of the inhibitor. In embodiments inhibition refers to reduction of a disease or symptoms of disease (e.g., cancer or an autoimmune disease). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits activity, e.g., by binding, partially or totally blocking, decreasing, preventing, delaying, inactivating, desensitizing, or down-regulating activity).

The terms “disease” or “condition” refer to a state of being or health status of a patient or subject capable of being treated with a compound, pharmaceutical composition, or method provided herein. In embodiments, the disease is cancer (e.g., lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). The disease may be an autoimmune, inflammatory, cancer, infectious, metabolic, developmental, cardiovascular, liver, intestinal, endocrine, neurological, or other disease.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head and neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer (e.g., leukemia, lymphoma, B cell lymphoma, or multiple myeloma)) means that the disease (e.g., cancer, (e.g., leukemia, lymphoma, B cell lymphoma, or multiple myeloma)) is caused by (in whole or in part), or a symptom of the disease is caused by (in whole or in part) the substance or substance activity or function.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

The term “effector cell ligand” as provided herein refers to a cell surface molecule expressed on an effector cell of the immune system (e.g., a cytotoxic T cell, a helper T cell, a B cell, a natural killer cell). Upon binding of the first antibody region to the effector cell ligand expressed on the effector cell, the effector cell is activated and able to exert its function (e.g., selective killing or eradication of malignant, infected or otherwise unhealthy cells). In embodiments, the effector cell ligand is a CD3 protein. In embodiments, the effector cell ligand is a CD16 protein. In embodiments, the effector cell ligand is a CD32 protein. In embodiments, the effector cell ligand is a NKp46 protein. The first antibody region as provided herein may be an antibody, an antibody variant, a fragment of an antibody or a fragment of an antibody variant.

A “CD3 protein” as referred to herein includes any of the recombinant or naturally-occurring forms of the Cluster of Differentiation 3 (CD3) proteins or variants or homologs thereof that comprise the CD3 complex that mediates signal transduction and maintains CD3 complex activity (e.g., within at least 50%, 80%, 90%, 95%, 96%, 97%, 98%, 99% or 100% activity compared to the CD3 complex). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring CD3 protein in the CD3 complex.

A “CD16 protein” as referred to herein includes any of the recombinant or naturally-occurring forms of the Cluster of Differentiation 16 (CD16) protein, also known as low affinity immunoglobulin gamma Fc region receptor III-A, or variants or homologs thereof that maintain CD16 activity (e.g. within at least 50%, 80%, 90%, 95%, 96%, 97%, 98%, 99% or 100% activity compared to CD16). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD16 protein. In embodiments, the CD16 protein is substantially identical to the protein identified by the UniProt reference number P08637 or a variant or homolog having substantial identity thereto.

A “CD32 protein” as referred to herein includes any of the recombinant or naturally-occurring forms of the Cluster of Differentiation 32 (CD32) protein, also known as low affinity immunoglobulin gamma Fc region receptor II-A, or variants or homologs thereof that maintain CD32 activity (e.g. within at least 50%, 80%, 90%, 95%, 96%, 97%, 98%, 99% or 100% activity compared to CD32). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD32 protein. In embodiments, the CD32 protein is substantially identical to the protein identified by the UniProt reference number P12318 or a variant or homolog having substantial identity thereto.

A “NKp46 protein” as referred to herein includes any of the recombinant or naturally-occurring forms of the NKp46 protein, also known as natural cytotoxicity triggering receptor 1, or variants or homologs thereof that maintain NKp46 activity (e.g. within at least 50%, 80%, 90%, 95%, 96%, 97%, 98%, 99% or 100% activity compared to NKp46). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring NKp46 protein. In embodiments, the NKp46 protein is substantially identical to the protein identified by the UniProt reference number 076036 or a variant or homolog having substantial identity thereto.

A “BAFF-R,” “BAFF receptor” or “BAFF-R protein” as referred to herein includes any of the recombinant or naturally-occurring forms of the B-cell activating factor receptor (BAFF-R) also known as tumor necrosis factor receptor superfamily member 13C (TNFRSF13C) or variants or homologs thereof that maintain BAFF-R activity (e.g., within at least 50%, 80%, 90%, 95%, 96%, 97%, 98%, 99% or 100% activity compared to BAFF-R). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring BAFF-R. In embodiments, the BAFF-R is substantially identical to the protein identified by the UniProt reference number Q96RJ3 or a variant or homolog having substantial identity thereto. In embodiments, the BAFF-R is substantially identical to the protein identified by the UniProt reference number Q9D8D0 or a variant or homolog having substantial identity thereto. In embodiments, the BAFF-R is substantially identical to the protein identified by the NCBI reference number GI: 16445027 or a variant or homolog having substantial identity thereto. In embodiments, the BAFF-R is substantially identical to the protein identified by the NCBI reference number GI: 16306481 or a variant or homolog having substantial identity thereto.

### II. Recombinant Protein Compositions

Provided herein are, inter alia, recombinant proteins (e.g., bispecific antibodies) capable of binding to BAFF-R and an effector cell through binding an effector-cell expressed antigen (e.g., CD3). The recombinant proteins (e.g., bispecific antibodies) include: (i) a first antibody region capable of binding an effector cell ligand (e.g., CD3); and (ii) a second antibody region capable of binding a second antibody antigen (e.g., human BAFF-R protein) and bind human BAFF-R protein with high affinity and specificity. The simultaneous binding of the recombinant proteins (e.g., bispecific antibodies) provided herein to an antigen on an effector cell (e.g., CD3 on effector T cells) and BAFF-R on B cells results in of the effector cell (e.g., T cell) and killing of malignant or autoreactive B cells. Thus, the compositions and methods provided herein may, inter alia, be used for the treatment of cancer (e.g., B cell malignancies) or autoimmune diseases. For the purpose of this invention the second antibody region provided herein including embodiments thereof includes any of the BAFF-R specific antibodies described in published International Application PCT/US2017/036178, which is hereby incorporated by reference and for all purposes. For example, the second antibody region may include any of the humanized BAFF-R antibodies referred to herein as “H90 Ab”, “humanized C90”, “humanized C90-1”, “humanized C90-2”, “humanized C90-3”, “C90”, “C90-1”, “C90-2”, or “C90-3.”

In one aspect, a recombinant protein including: (i) a first antibody region capable of binding an effector cell ligand (e.g., CD3); and (ii) a second antibody region including: (a) a light chain variable domain and a heavy chain variable domain is provided. The light chain variable domain includes a CDR L1 as set forth in SEQ ID NO: 1, a CDR L2 as set forth in SEQ ID NO:2 and a CDR L3 as set forth in SEQ ID NO:3. The heavy chain variable domain includes a CDR H1 as set forth in SEQ ID NO:4, a CDR H2 as set forth in SEQ ID NO:5, and a CDR H3 as set forth in SEQ ID NO:6. In embodiments, the second antibody region binds a BAFF-R protein or fragment thereof. Wherein a CDR as proved herein is “set forth in” a SEQ ID NO (e.g., SEQ ID NO 1, 2, 3, 4, 5, 6, 38, 38, 40, 41, 42, 43), the CDR has the sequence referred to by that SEQ ID NO. Generally, if a sequence is set forth in a sequence of a particular SEQ ID NO, said sequence has the sequence of the sequence referred to by said SEQ ID NO.

The recombinant proteins provided herein may include light chain and heavy chain variable regions derived from different antibody clones (e.g., C90-1, C90-2, C90-3, C55-1, C55-2, C55-3), which include the same CDRs and different FRs (e.g., C90-1, C90-2 and C90-3) or different CDRs (e.g., CDRs from C90 clones and CDRs from C55 clones). The second antibody region of the recombinant protein provided herein including embodiments thereof may be an scFv. Where the second antibody region is an scFv, the second antibody region may be referred to as H90, C90-1, C90-2 or C90-3 and include a CDR L1 as set forth in SEQ ID NO: 1, a CDR L2 as set forth in SEQ ID NO:2, a CDR L3 as set forth in SEQ ID NO:3, a CDR H1 as set forth in SEQ ID NO:4, a CDR H2 as set forth in SEQ ID NO:5, and a CDR H3 as set forth in SEQ ID NO:6. In embodiments, the second antibody region is an scFv and is referred to as C55-1, C55-2 or C55-3 and includes a CDR L1 as set forth in SEQ ID NO:38, a CDR L2 as set forth in SEQ ID NO:39, a CDR L3 as set forth in SEQ ID NO:40, a CDR H1 as set forth in SEQ ID NO:41, a CDR H2 as set forth in SEQ ID NO:42, and a CDR H3 as set forth in SEQ ID NO:43.

In embodiments, the light chain variable domain includes the sequence of SEQ ID NO:7, SEQ ID NO:8 or SEQ ID NO:9. In embodiments, the light chain variable domain includes the sequence of SEQ ID NO:7. In embodiments, the light chain variable domain includes the sequence of SEQ ID NO:8. In embodiments, the light chain variable domain includes the sequence of SEQ ID NO:9. In embodiments, the light chain variable domain is the sequence of SEQ ID NO:7. In embodiments, the light chain variable domain is the sequence of SEQ ID NO:8. In embodiments, the light chain variable domain is the sequence of SEQ ID NO:9.

In embodiments, the heavy chain variable domain includes the sequence of SEQ ID NO:10, SEQ ID NO:11 or SEQ ID NO:12. In embodiments, the heavy chain variable domain includes the sequence of SEQ ID NO: 10. In embodiments, the heavy chain variable domain includes the sequence of SEQ ID NO: 11. In embodiments, the heavy chain variable domain includes the sequence of SEQ ID NO: 12. In embodiments, the heavy chain variable domain is the sequence of SEQ ID NO: 10. In embodiments, the heavy chain variable domain is the sequence of SEQ ID NO: 11. In embodiments, the heavy chain variable domain is the sequence of SEQ ID NO:12.

In embodiments, the light chain variable domain includes a FR L1 as set forth in SEQ ID NO: 13, a FR L2 as set forth in SEQ ID NO: 14, FR L3 as set forth in SEQ ID NO: 15 and a FR L4 as set forth in SEQ ID NO: 16. In embodiments, the heavy chain variable domain includes a FR H1 as set forth in SEQ ID NO:25, a FR H2 as set forth in SEQ ID NO:26, FR H3 as set forth in SEQ ID NO:27 and a FR H4 as set forth in SEQ ID NO:28.

In embodiments, the light chain variable domain includes a FR L1 as set forth in SEQ ID NO:17, a FR L2 as set forth in SEQ ID NO: 18, FR L3 as set forth in SEQ ID NO:19 and a FR L4 as set forth in SEQ ID NO:20. In embodiments, the heavy chain variable domain includes a FR H1 as set forth in SEQ ID NO:29, a FR H2 as set forth in SEQ ID NO:30, FR H3 as set forth in SEQ ID NO:31 and a FR H4 as set forth in SEQ ID NO:32.

In embodiments, the light chain variable domain includes a FR L1 as set forth in SEQ ID NO:21, a FR L2 as set forth in SEQ ID NO:22, FR L3 as set forth in SEQ ID NO:23 and a FR L4 as set forth in SEQ ID NO:24. In embodiments, the heavy chain variable domain includes a FR H1 as set forth in SEQ ID NO:33, a FR H2 as set forth in SEQ ID NO:34, FR H3 as set forth in SEQ ID NO:35 and a FR H4 as set forth in SEQ ID NO:36.

In embodiments, the second antibody region includes a light chain variable domain of SEQ ID NO:7 and a heavy chain variable region of SEQ ID NO: 10. In embodiments, the second antibody region is C90-1. In further embodiments, the second antibody region includes a light chain variable domain of SEQ ID NO:7 and a heavy chain variable region of SEQ ID NO: 10.

In embodiments, the second antibody region includes a light chain variable domain of SEQ ID NO:8 and a heavy chain variable region of SEQ ID NO: 11. In embodiments, the second antibody region is C90-2. In further embodiments, the second antibody region includes a light chain variable domain of SEQ ID NO:8 and a heavy chain variable region of SEQ ID NO: 11.

In embodiments, the second antibody region includes a light chain variable domain of SEQ ID NO:9 and a heavy chain variable region of SEQ ID NO: 12. In embodiments, the second antibody region is C90-3. In further embodiments, the second antibody region includes a light chain variable domain of SEQ ID NO:9 and a heavy chain variable region of SEQ ID NO: 12.

In one aspect, a recombinant protein including: (i) a first antibody region capable of binding an effector cell ligand; and (ii) a second antibody region including: (a) a light chain variable domain and a heavy chain variable domain is provided. The light chain variable domain includes a CDR L1 as set forth in SEQ ID NO:38, a CDR L2 as set forth in SEQ ID NO:39 and a CDR L3 as set forth in SEQ ID NO:40. The heavy chain variable domain includes a CDR H1 as set forth in SEQ ID NO:41, a CDR H2 as set forth in SEQ ID NO:42, and a CDR H3 as set forth in SEQ ID NO:43.

In embodiments, the light chain variable domain includes the sequence of SEQ ID NO:44, SEQ ID NO:45 or SEQ ID NO:46. In embodiments, the light chain variable domain includes the sequence of SEQ ID NO:44. In embodiments, the light chain variable domain includes the sequence of SEQ ID NO:45. In embodiments, the light chain variable domain includes the sequence of SEQ ID NO:46. In embodiments, the light chain variable domain is the sequence of SEQ ID NO:44. In embodiments, the light chain variable domain is the sequence of SEQ ID NO:45. In embodiments, the light chain variable domain is the sequence of SEQ ID NO:46.

In embodiments, the heavy chain variable domain includes the sequence of SEQ ID NO:47, SEQ ID NO:48 or SEQ ID NO:49. In embodiments, the heavy chain variable domain includes the sequence of SEQ ID NO:47. In embodiments, the heavy chain variable domain includes the sequence of SEQ ID NO:48. In embodiments, the heavy chain variable domain includes the sequence of SEQ ID NO:49. In embodiments, the heavy chain variable domain is the sequence of SEQ ID NO:47. In embodiments, the heavy chain variable domain is the sequence of SEQ ID NO:48. In embodiments, the heavy chain variable domain is the sequence of SEQ ID NO:49.

In embodiments, the light chain variable domain includes a FR L1 as set forth in SEQ ID NO:50, a FR L2 as set forth in SEQ ID NO:51, FR L3 as set forth in SEQ ID NO:52 and a FR L4 as set forth in SEQ ID NO:53. In embodiments, the heavy chain variable domain includes a FR H1 as set forth in SEQ ID NO:62, a FR H2 as set forth in SEQ ID NO:63, FR H3 as set forth in SEQ ID NO:64 and a FR H4 as set forth in SEQ ID NO:65.

In embodiments, the light chain variable domain includes a FR L1 as set forth in SEQ ID NO:54, a FR L2 as set forth in SEQ ID NO:55, FR L3 as set forth in SEQ ID NO:56 and a FR L4 as set forth in SEQ ID NO:57. In embodiments, the heavy chain variable domain includes a FR H1 as set forth in SEQ ID NO:66, a FR H2 as set forth in SEQ ID NO:67, FR H3 as set forth in SEQ ID NO:68 and a FR H4 as set forth in SEQ ID NO:69.

In embodiments, the light chain variable domain includes a FR L1 as set forth in SEQ ID NO:58, a FR L2 as set forth in SEQ ID NO:59, FR L3 as set forth in SEQ ID NO:60 and a FR L4 as set forth in SEQ ID NO:61. In embodiments, the heavy chain variable domain includes a FR H1 as set forth in SEQ ID NO:70, a FR H2 as set forth in SEQ ID NO:71, FR H3 as set forth in SEQ ID NO:72 and a FR H4 as set forth in SEQ ID NO:73.

In embodiments, the second antibody region includes a light chain variable domain of SEQ ID NO:44 and a heavy chain variable region of SEQ ID NO:47. In embodiments, the second antibody region is C55-1. In further embodiments, the second antibody region includes a light chain variable domain of SEQ ID NO:44 and a heavy chain variable region of SEQ ID NO:47.

In embodiments, the second antibody region includes a light chain variable domain of SEQ ID NO:45 and a heavy chain variable region of SEQ ID NO:48. In embodiments, the second antibody region is C55-2. In further embodiments, the second antibody region includes a light chain variable domain of SEQ ID NO:45 and a heavy chain variable region of SEQ ID NO:48.

In embodiments, the second antibody region includes a light chain variable domain of SEQ ID NO:46 and a heavy chain variable region of SEQ ID NO:49. In embodiments, the second antibody region is C55-3. In further embodiments, the second antibody region includes a light chain variable domain of SEQ ID NO:46 and a heavy chain variable region of SEQ ID NO:49.

The position of CDRs and FRs may be defined by the Kabat numbering system (Kabat et al., Sequences of Proteins of Immunological Interest, Fifth Edition, U.S. Department of Health and Human Services, U.S. Government Printing Office (1991)). Likewise, the positions occupied by individual residues within the light or the heavy chain of an antibody may be defined by the Kabat numbering system. Therefore, the location of residues required for binding within a light chain and a heavy chain of a humanized antibody may be defined by the position of the residue according to the Kabat numbering system as is well known in the art. As described above, a humanized antibody may be an antibody having CDRs from a donor antibody (e.g., mouse) and variable region framework (FR) from a human antibody. The framework regions (FRs) are said to hold the CDRs in place in a humanized antibody. Proceeding from the amino-terminus, these regions are designated FR L1, FR L2, FR L3, and FR L4 for the light chain and FR H1, FR H2, FR H3, and FR H4, for the heavy chain, respectively. Surprisingly, the present invention provides for recombinant proteins (e.g., bispecific antibodies) that include one or more residues within the framework regions that are important for epitope binding of the second antibody region. A framework region residue involved in (or important for) epitope binding (e.g., BAFF-R binding) is referred to herein as a binding framework region residue. The binding framework region residues may reside in the framework region of a light chain variable domain (i.e., FR L1, FR L2, FR L3, FR L4) or they may reside in the framework of a heavy chain variable domain (i.e., FR H1, FR H2, FR H3, FR H4). A binding framework residue residing in the FR L3 region of a light chain is referred to herein as a FR L3 binding framework region residue. Thus, a binding framework region residue residing in the FR H3 region of a heavy chain is referred to herein as a FR H3 binding framework region residue.

In embodiments, the second antibody region includes at least one binding framework region residue. In embodiments, the light chain variable domain includes at least one binding framework region residue. In embodiments, the light chain variable domain includes one or more FR L1, FR L2, FR L3 or FR L4 binding framework region residues. In embodiments, the light chain variable domain includes one or more FR L1 binding framework region residues. In embodiments, the light chain variable domain includes one or more FR L2 binding framework region residues. In embodiments, the light chain variable domain includes one or more FR L3 binding framework region residues. In embodiments, the light chain variable domain includes one or more FR L4 binding framework region residues. In embodiments, the heavy chain variable domain includes one or more FR H1, FR H2, FR H3 or FR H4 binding framework region residues. In embodiments, the heavy chain variable domain includes one or more FR H1 binding framework region residues. In embodiments, the heavy chain variable domain includes one or more FR H2 binding framework region residues. In embodiments, the heavy chain variable domain includes one or more FR H3 binding framework region residues. In embodiments, the heavy chain variable domain includes one or more FR H4 binding framework region residues.

In embodiments, the light chain variable domain includes at least one binding framework region residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42, 43, 44, 45, 46, 47, 48, 49, 50 or more residues) and the heavy chain variable domain includes at least one binding framework region residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42, 43, 44, 45, 46, 47, 48, 49, 50 or more residues). The position of a binding framework region residue within an antibody or antibody region may be defined by the Kabat numbering system similar to the positions of CDR residues.

In embodiments, the light chain variable domain includes a serine at a position corresponding to Kabat position 7. In embodiments, the light chain variable domain includes a proline at a position corresponding to Kabat position 8. In embodiments, the light chain variable domain includes a valine at a position corresponding to Kabat position 15. In embodiments, the light chain variable domain includes a threonine at a position corresponding to Kabat position 22. In embodiments, the light chain variable domain includes a glutamine at a position corresponding to Kabat position 24. In embodiments, the light chain variable domain includes a glycine at a position corresponding to Kabat position 41. In embodiments, the light chain variable domain includes a lysine at a position corresponding to Kabat position 42. In embodiments, the light chain variable domain includes an alanine at a position corresponding to Kabat position 43. In embodiments, the light chain variable domain includes a proline at a position corresponding to Kabat position 44. In embodiments, the light chain variable domain includes a threonine at a position corresponding to Kabat position 56. In embodiments, the light chain variable domain includes a threonine at a position corresponding to Kabat position 72. In embodiments, the light chain variable domain includes a phenylalanine at a position corresponding to Kabat position 73. In embodiments, the light chain variable domain includes a glutamine at a position corresponding to Kabat position 79. In embodiments, the light chain variable domain includes a valine at a position corresponding to Kabat position 104.

In embodiments, the light chain variable domain includes a serine at a position corresponding to Kabat position 7, a proline at a position corresponding to Kabat position 8, a valine at a position corresponding to Kabat position 15, a threonine at a position corresponding to Kabat position 22, a glutamine or a serine at a position corresponding to Kabat position 24, a glycine at a position corresponding to Kabat position 41, a lysine at a position corresponding to Kabat position 42, an alanine or a threonine at a position corresponding to Kabat position 43, a proline at a position corresponding to Kabat position 44, a threonine at a position corresponding to Kabat position 56, a threonine at a position corresponding to Kabat position 72, a phenylalanine or a lysine at a position corresponding to Kabat position 73, a glutamine at a position corresponding to Kabat position 79 or a valine at a position corresponding to Kabat position 104.

In embodiments, the light chain variable domain includes a serine at a position corresponding to Kabat position 7, a proline at a position corresponding to Kabat position 8, a valine at a position corresponding to Kabat position 15, a threonine at a position corresponding to Kabat position 22, a glutamine or a serine at a position corresponding to Kabat position 24, a glycine at a position corresponding to Kabat position 41, a lysine at a position corresponding to Kabat position 42, an alanine or a threonine at a position corresponding to Kabat position 43, a proline at a position corresponding to Kabat position 44, a threonine at a position corresponding to Kabat position 56, a threonine at a position corresponding to Kabat position 72, a phenylalanine or a lysine at a position corresponding to Kabat position 73, a glutamine at a position corresponding to Kabat position 79 and a valine at a position corresponding to Kabat position 104.

In embodiments, the light chain variable domain includes a binding framework region residue that is a serine at a position corresponding to Kabat position 7, a proline at a position corresponding to Kabat position 8, a valine at a position corresponding to Kabat position 15, a threonine at a position corresponding to Kabat position 22, a glutamine or a serine at a position corresponding to Kabat position 24, a glycine at a position corresponding to Kabat position 41, a lysine at a position corresponding to Kabat position 42, an alanine or a threonine at a position corresponding to Kabat position 43, a proline at a position corresponding to Kabat position 44, a threonine at a position corresponding to Kabat position 56, a threonine at a position corresponding to Kabat position 72, a phenylalanine or a lysine at a position corresponding to Kabat position 73, a glutamine at a position corresponding to Kabat position 79 or a valine at a position corresponding to Kabat position 104.

In embodiments, the heavy chain variable domain on includes a threonine or an alanine at a position corresponding to Kabat position 10. In embodiments, the heavy chain variable domain includes a lysine at a position corresponding to Kabat position 11. In embodiments, the heavy chain variable domain includes a valine at a position corresponding to Kabat position 12. In embodiments, the heavy chain variable domain includes a threonine at a position corresponding to Kabat position 15. In embodiments, the heavy chain variable domain includes a threonine at a position corresponding to Kabat position 19. In embodiments, the heavy chain variable domain includes a threonine at a position corresponding to Kabat position 23. In embodiments, the heavy chain variable domain includes a proline at a position corresponding to Kabat position 41. In embodiments, the heavy chain variable domain includes an alanine at a position corresponding to Kabat position 44. In embodiments, the heavy chain variable domain includes a proline or a threonine at a position corresponding to Kabat position 61. In embodiments, the heavy chain variable domain includes an arginine at a position corresponding to Kabat position 66. In embodiments, the heavy chain variable domain includes a threonine at a position corresponding to Kabat position 70. In embodiments, the heavy chain variable domain includes a lysine at a position corresponding to Kabat position 75. In embodiments, the heavy chain variable domain includes a valine at a position corresponding to Kabat position 79. In embodiments, the heavy chain variable domain includes a threonine at a position corresponding to Kabat position 81. In embodiments, the heavy chain variable domain includes a methionine at a position corresponding to Kabat position 82. In embodiments, the heavy chain variable domain includes an asparagine at a position corresponding to Kabat position 82B. In embodiments, the heavy chain variable domain includes a methionine at a position corresponding to Kabat position 82C. In embodiments, the heavy chain variable domain includes a proline at a position corresponding to Kabat position 84. In embodiments, the heavy chain variable domain includes a valine at a position corresponding to Kabat position 85. In embodiments, the heavy chain variable domain includes a lysine at a position corresponding to Kabat position 108. In embodiments, the heavy chain variable domain includes a valine at a position corresponding to Kabat position 109.

In embodiments, the heavy chain variable domain includes a threonine or an alanine at a position corresponding to Kabat position 10, a lysine at a position corresponding to Kabat position 11, a valine at a position corresponding to Kabat position 12, a threonine at a position corresponding to Kabat position 15, a threonine at a position corresponding to Kabat position 19, a threonine at a position corresponding to Kabat position 23, a proline at a position corresponding to Kabat position 41, an alanine at a position corresponding to Kabat position 44, a proline, a serine or a threonine at a position corresponding to Kabat position 61, an arginine at a position corresponding to Kabat position 66, a threonine at a position corresponding to Kabat position 70, a lysine at a position corresponding to Kabat position 75, a valine at a position corresponding to Kabat position 79, a threonine or a lysine at a position corresponding to Kabat position 81, a methionine at a position corresponding to Kabat position 82, an asparagine at a position corresponding to Kabat position 82B, a methionine at a position corresponding to Kabat position 82C, a proline at a position corresponding to Kabat position 84, a valine at a position corresponding to Kabat position 85, a lysine at a position corresponding to Kabat position 108 or a valine at a position corresponding to Kabat position 109.

In embodiments, the heavy chain variable domain includes a threonine or an alanine at a position corresponding to Kabat position 10, a lysine at a position corresponding to Kabat position 11, a valine at a position corresponding to Kabat position 12, a threonine at a position corresponding to Kabat position 15, a threonine at a position corresponding to Kabat position 19, a threonine at a position corresponding to Kabat position 23, a proline at a position corresponding to Kabat position 41, an alanine at a position corresponding to Kabat position 44, a proline, a serine or a threonine at a position corresponding to Kabat position 61, an arginine at a position corresponding to Kabat position 66, a threonine at a position corresponding to Kabat position 70, a lysine at a position corresponding to Kabat position 75, a valine at a position corresponding to Kabat position 79, a threonine or a lysine at a position corresponding to Kabat position 81, a methionine at a position corresponding to Kabat position 82, an asparagine at a position corresponding to Kabat position 82B, a methionine at a position corresponding to Kabat position 82C, a proline at a position corresponding to Kabat position 84, a valine at a position corresponding to Kabat position 85, a lysine at a position corresponding to Kabat position 108 and a valine at a position corresponding to Kabat position 109.

In embodiments, the heavy chain variable domain includes a binding framework region residue that is a threonine or an alanine at a position corresponding to Kabat position 10, a lysine at a position corresponding to Kabat position 11, a valine at a position corresponding to Kabat position 12, a threonine at a position corresponding to Kabat position 15, a threonine at a position corresponding to Kabat position 19, a threonine at a position corresponding to Kabat position 23, a proline at a position corresponding to Kabat position 41, an alanine at a position corresponding to Kabat position 44, a proline, a serine or a threonine at a position corresponding to Kabat position 61, an arginine at a position corresponding to Kabat position 66, a threonine at a position corresponding to Kabat position 70, a lysine at a position corresponding to Kabat position 75, a valine at a position corresponding to Kabat position 79, a threonine or a lysine at a position corresponding to Kabat position 81, a methionine at a position corresponding to Kabat position 82, an asparagine at a position corresponding to Kabat position 82B, a methionine at a position corresponding to Kabat position 82C, a proline at a position corresponding to Kabat position 84, a valine at a position corresponding to Kabat position 85, a lysine at a position corresponding to Kabat position 108 or a valine at a position corresponding to Kabat position 109.

The recombinant proteins (e.g., bispecific antibodies) provided herein including embodiments thereof may include one or more Fab′ fragments. The recombinant proteins (e.g., bispecific antibodies) provided herein including embodiments include a first antibody region and a second antibody region, wherein the first antibody region may be a first Fab′ fragment or the second antibody region may be a second Fab′ fragment. Where the recombinant protein (e.g., bispecific antibodies) provided herein includes a Fab′ fragment, the recombinant protein may include a first antibody binding region and a second antibody binding region each independently including a heavy chain (e.g., including a constant and a variable region) and a light chain (e.g., including a constant and a variable region). In embodiments, the Fab′ fragment includes a humanized heavy chain (e.g., including a constant and a variable region) and a humanized light chain (e.g., including a constant and a variable region). Thus, in embodiments, the first antibody region is a first Fab′ fragment or the second antibody region is a second Fab′ fragment. In embodiments, the first antibody region is a first Fab′ fragment. In embodiments, the second antibody region is a second Fab′ fragment. In embodiments, the first antibody region is a first Fab′ fragment and the second antibody region is a second Fab′ fragment.

The first or second antibody region may be a first scFv or a second scFv, respectively. A single chain antibody includes a variable light chain and a variable heavy chain. A person of skill in the art will immediately recognize that a single chain antibody includes a single light chain and a single heavy chain, in contrast to an immunoglobulin antibody, which includes two identical pairs of polypeptide chains, each pair having one light chain and one heavy chain. Each light chain and heavy chain in turn consists of two regions: a variable (“V”) region (i.e., variable light chain and variable heavy chain) involved in binding the target antigen, and a constant (“C”) region that interacts with other components of the immune system. The variable light chain and the variable heavy chain in a single chain antibody may be linked through a linker peptide. In embodiments, the linker peptide includes the sequence as set forth in SEQ ID NO:37. In embodiments, the linker peptide is the sequence as set forth in SEQ ID NO:37. Examples for linker peptides of single chain antibodies are described in Bird, R. E., Hardman, K. D., Jacobson, J. W., Johnson, S., Kaufman, B. M., Lee, S. M., Lee, T., Pope, S. H., Riordan, G. S. and Whitlow, M. (1988). Methods of making scFv antibodies have been described. See, Huse et al., Science 246:1275-1281 (1989); Ward et al., Nature 341:544-546 (1989); and Vaughan et al., Nature Biotech. 14:309-314 (1996). Briefly, mRNA from B-cells from an immunized animal is isolated and cDNA is prepared. The cDNA is amplified using primers specific for the variable domains of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid, which encodes the scFv, is inserted into a vector and expressed in the appropriate host cell.

In embodiments, the first antibody region is a first single chain variable fragment or the second antibody region is a second single chain variable fragment. In embodiments, the first antibody region is a first single chain variable fragment. In embodiments, the second antibody region is a second single chain variable fragment. In embodiments, the first antibody region is a first single chain variable fragment and the second antibody region is a second single chain variable fragment.

The ability of an antibody to bind a specific epitope (e.g., BAFF-R) can be described by the equilibrium dissociation constant (K_{D}). The equilibrium dissociation constant (K_{D}) as defined herein is the ratio of the dissociation rate (K-off) and the association rate (K-on) of the second antibody region capable of binding a BAFF-R protein. It is described by the following formula: K_{D}=K−off/K−on. Thus, in embodiments, the second antibody region is capable of binding a BAFF-R protein with an equilibrium dissociation constant (K_{D}) of less than about 5 nM. In embodiments, the second antibody region is capable of binding a BAFF-R protein with an equilibrium dissociation constant (K_{D}) of less than about 4.5 nM. In embodiments, the Second antibody region is capable of binding a BAFF-R protein with an equilibrium dissociation constant (K_{D}) of less than about 4 nM. In embodiments, the second antibody region is capable of binding a BAFF-R protein with an equilibrium dissociation constant (K_{D}) of less than about 3.5 nM. In embodiments, the second antibody region is capable of binding a BAFF-R protein with an equilibrium dissociation constant (K_{D}) of less than about 3 nM. In embodiments, the second antibody region is capable of binding a BAFF-R protein with an equilibrium dissociation constant (K_{D}) of less than about 2.5 nM. In embodiments, the second antibody region is capable of binding a BAFF-R protein with an equilibrium dissociation constant (K_{D}) of less than about 2 nM. In embodiments, the second antibody region is capable of binding a BAFF-R protein with an equilibrium dissociation constant (K_{D}) of less than about 1.5 nM. In embodiments, the second antibody region is capable of binding a BAFF-R protein with an equilibrium dissociation constant (K_{D}) of less than about 1 nM. In embodiments, the second antibody region is capable of binding a BAFF-R protein with an equilibrium dissociation constant (K_{D}) of less than about 0.5 nM.

In embodiments, the second antibody region is capable of binding a BAFF-R protein with an equilibrium dissociation constant (K_{D}) of about 0.5 nM. In embodiments, the second antibody region is capable of binding a BAFF-R protein with an equilibrium dissociation constant (K_{D}) of about 1 nM. In embodiments, the second antibody region is capable of binding a BAFF-R protein with an equilibrium dissociation constant (K_{D}) of about 1.5 nM. In embodiments, the second antibody region is capable of binding a BAFF-R protein with an equilibrium dissociation constant (K_{D}) of about 2 nM. In embodiments, the second antibody region is capable of binding a BAFF-R protein with an equilibrium dissociation constant (K_{D}) of about 2.5 nM. In embodiments, the second antibody region is capable of binding a BAFF-R protein with an equilibrium dissociation constant (K_{D}) of about 3 nM. In embodiments, the second antibody region is capable of binding a BAFF-R protein with an equilibrium dissociation constant (K_{D}) of about 3.5 nM. In embodiments, the second antibody region is capable of binding a BAFF-R protein with an equilibrium dissociation constant (K_{D}) of about 4 nM. In embodiments, the second antibody region is capable of binding a BAFF-R protein with an equilibrium dissociation constant (K_{D}) of about 4.5 nM. In embodiments, the second antibody region is capable of binding a BAFF-R protein with an equilibrium dissociation constant (K_{D}) of about 5 nM. In embodiments, the second antibody region is capable of binding a BAFF-R protein with an equilibrium dissociation constant (K_{D}) of about 0.5, 1, 1.5, 2, 2.5, 3, 3.5, 4, 4.5, or 5 nM.

In one aspect, a recombinant protein capable of binding BAFF-R with a K_{D} of less than about 4 nM is provided. In another aspect, a recombinant protein bound to a BAFF-R at a K_{D} of less than about 4 nM is provided. In embodiments, the antibody does not induce BAFF-R activity.

The recombinant protein provided herein includes a first antibody region capable of binding an effector cell ligand. In embodiments, the effector cell ligand is a CD3 protein. In embodiments, the first antibody region includes: (a) a light chain variable domain including a CDR L1 as set forth in SEQ ID NO:74, a CDR L2 as set forth in SEQ ID NO:75 and a CDR L3 as set forth in SEQ ID NO:76; and (b) a heavy chain variable domain including a CDR H1 as set forth in SEQ ID NO:77, a CDR H2 as set forth in SEQ ID NO:78 and a CDR H3 as set forth in SEQ ID NO:79.

In embodiments, the first antibody region includes: (a) a light chain variable domain including a CDR L1 as set forth in SEQ ID NO:81, a CDR L2 as set forth in SEQ ID NO:82 and a CDR L3 as set forth in SEQ ID NO:83; and (b) a heavy chain variable domain including a CDR H1 as set forth in SEQ ID NO:84, a CDR H2 as set forth in SEQ ID NO:85 and a CDR H3 as set forth in SEQ ID NO:86.

In embodiments, the first antibody region is a first single chain variable fragment (scFv). In embodiments, the first scFv includes the sequence of SEQ ID NO:80 or SEQ ID NO:87. In embodiments, the first scFv includes the sequence of SEQ ID NO:80. In embodiments, the first scFv includes the sequence of SEQ ID NO:87. In embodiments, the first scFv is the sequence of SEQ ID NO:80 or SEQ ID NO:87. In embodiments, the first scFv is the sequence of SEQ ID NO:80. In embodiments, the first scFv is the sequence of SEQ ID NO:87.

In embodiments, the second antibody region is bound to a BAFF-R protein. In embodiments, the BAFF-R protein is a human BAFF-R protein. In embodiments, the BAFF-R protein is encoded by a nucleic acid sequence identified by NCBI Gene ID number 115650. In embodiments, the BAFF-R protein forms part of a cell. In embodiments, the BAFF-R protein is expressed on the surface of the cell. In embodiments, the cell is a lymphoid cell. In embodiments, the cell is a B cell. In embodiments, the cell is a cancer cell. In embodiments, the cancer cell is a lymphoma cell.

In one embodiment, the recombinant protein includes a first antibody region including (a) a light chain variable domain including a CDR L1 as set forth in SEQ ID NO:74, a CDR L2 as set forth in SEQ ID NO:75 and a CDR L3 as set forth in SEQ ID NO:76; and (b) a heavy chain variable domain including a CDR H1 as set forth in SEQ ID NO:77, a CDR H2 as set forth in SEQ ID NO:78 and a CDR H3 as set forth in SEQ ID NO:79 and a second antibody region including a light chain variable domain that has the sequence of SEQ ID NO:7 and a heavy chain variable domain that has the sequence of SEQ ID NO: 10.

In one embodiment, the recombinant protein includes a first antibody region including (a) a light chain variable domain including a CDR L1 as set forth in SEQ ID NO:74, a CDR L2 as set forth in SEQ ID NO:75 and a CDR L3 as set forth in SEQ ID NO:76; and (b) a heavy chain variable domain including a CDR H1 as set forth in SEQ ID NO:77, a CDR H2 as set forth in SEQ ID NO:78 and a CDR H3 as set forth in SEQ ID NO:79 and a second antibody region including a light chain variable domain that has the sequence of SEQ ID NO:8 and a heavy chain variable domain that has the sequence of SEQ ID NO: 11.

In one embodiment, the recombinant protein includes a first antibody region including (a) a light chain variable domain including a CDR L1 as set forth in SEQ ID NO:74, a CDR L2 as set forth in SEQ ID NO:75 and a CDR L3 as set forth in SEQ ID NO:76; and (b) a heavy chain variable domain including a CDR H1 as set forth in SEQ ID NO:77, a CDR H2 as set forth in SEQ ID NO:78 and a CDR H3 as set forth in SEQ ID NO:79 and a second antibody region including a light chain variable domain that has the sequence of SEQ ID NO:9 and a heavy chain variable domain that has the sequence of SEQ ID NO: 12.

In one embodiment, the recombinant protein includes a first antibody region including (a) a light chain variable domain including a CDR L1 as set forth in SEQ ID NO:81, a CDR L2 as set forth in SEQ ID NO:82 and a CDR L3 as set forth in SEQ ID NO:83; and (b) a heavy chain variable domain including a CDR H1 as set forth in SEQ ID NO:84, a CDR H2 as set forth in SEQ ID NO:85 and a CDR H3 as set forth in SEQ ID NO:86; and a second antibody region including a light chain variable domain that has the sequence of SEQ ID NO:7 and a heavy chain variable domain that has the sequence of SEQ ID NO: 10.

In one embodiment, the recombinant protein includes a first antibody region including (a) a light chain variable domain including a CDR L1 as set forth in SEQ ID NO:81, a CDR L2 as set forth in SEQ ID NO:82 and a CDR L3 as set forth in SEQ ID NO:83; and (b) a heavy chain variable domain including a CDR H1 as set forth in SEQ ID NO:84, a CDR H2 as set forth in SEQ ID NO:85 and a CDR H3 as set forth in SEQ ID NO:86; and a second antibody region including a light chain variable domain that has the sequence of SEQ ID NO:8 and a heavy chain variable domain that has the sequence of SEQ ID NO: 11.

In one embodiment, the recombinant protein includes a first antibody region including (a) a light chain variable domain including a CDR L1 as set forth in SEQ ID NO:81, a CDR L2 as set forth in SEQ ID NO:82 and a CDR L3 as set forth in SEQ ID NO:83; and (b) a heavy chain variable domain including a CDR H1 as set forth in SEQ ID NO:84, a CDR H2 as set forth in SEQ ID NO:85 and a CDR H3 as set forth in SEQ ID NO:86; and a second antibody region including a light chain variable domain that has the sequence of SEQ ID NO:9 and a heavy chain variable domain that has the sequence of SEQ ID NO: 12.

In one embodiment, the recombinant protein includes a first antibody region including (a) a light chain variable domain including a CDR L1 as set forth in SEQ ID NO:74, a CDR L2 as set forth in SEQ ID NO:75 and a CDR L3 as set forth in SEQ ID NO:76; and (b) a heavy chain variable domain including a CDR H1 as set forth in SEQ ID NO:77, a CDR H2 as set forth in SEQ ID NO:78 and a CDR H3 as set forth in SEQ ID NO:79 and a second antibody region including a light chain variable domain that has the sequence of SEQ ID NO:44 and a heavy chain variable domain that has the sequence of SEQ ID NO:47.

In one embodiment, the recombinant protein includes a first antibody region including (a) a light chain variable domain including a CDR L1 as set forth in SEQ ID NO:74, a CDR L2 as set forth in SEQ ID NO:75 and a CDR L3 as set forth in SEQ ID NO:76; and (b) a heavy chain variable domain including a CDR H1 as set forth in SEQ ID NO:77, a CDR H2 as set forth in SEQ ID NO:78 and a CDR H3 as set forth in SEQ ID NO:79 and a second antibody region including a light chain variable domain that has the sequence of SEQ ID NO:45 and a heavy chain variable domain that has the sequence of SEQ ID NO:48.

In one embodiment, the recombinant protein includes a first antibody region including (a) a light chain variable domain including a CDR L1 as set forth in SEQ ID NO:74, a CDR L2 as set forth in SEQ ID NO:75 and a CDR L3 as set forth in SEQ ID NO:76; and (b) a heavy chain variable domain including a CDR H1 as set forth in SEQ ID NO:77, a CDR H2 as set forth in SEQ ID NO:78 and a CDR H3 as set forth in SEQ ID NO:79 and a second antibody region including a light chain variable domain that has the sequence of SEQ ID NO:46 and a heavy chain variable domain that has the sequence of SEQ ID NO:49.

In one embodiment, the recombinant protein includes a first antibody region including (a) a light chain variable domain including a CDR L1 as set forth in SEQ ID NO:81, a CDR L2 as set forth in SEQ ID NO:82 and a CDR L3 as set forth in SEQ ID NO:83; and (b) a heavy chain variable domain including a CDR H1 as set forth in SEQ ID NO:84, a CDR H2 as set forth in SEQ ID NO:85 and a CDR H3 as set forth in SEQ ID NO:86; and a second antibody region including a light chain variable domain that has the sequence of SEQ ID NO:44 and a heavy chain variable domain that has the sequence of SEQ ID NO:47.

In one embodiment, the recombinant protein includes a first antibody region including (a) a light chain variable domain including a CDR L1 as set forth in SEQ ID NO:81, a CDR L2 as set forth in SEQ ID NO:82 and a CDR L3 as set forth in SEQ ID NO:83; and (b) a heavy chain variable domain including a CDR H1 as set forth in SEQ ID NO:84, a CDR H2 as set forth in SEQ ID NO:85 and a CDR H3 as set forth in SEQ ID NO:86; and a second antibody region including a light chain variable domain that has the sequence of SEQ ID NO:45 and a heavy chain variable domain that has the sequence of SEQ ID NO:48.

In one embodiment, the recombinant protein includes a first antibody region including (a) a light chain variable domain including a CDR L1 as set forth in SEQ ID NO:81, a CDR L2 as set forth in SEQ ID NO:82 and a CDR L3 as set forth in SEQ ID NO:83; and (b) a heavy chain variable domain including a CDR H1 as set forth in SEQ ID NO:84, a CDR H2 as set forth in SEQ ID NO:85 and a CDR H3 as set forth in SEQ ID NO:86; and a second antibody region including a light chain variable domain that has the sequence of SEQ ID NO:46 and a heavy chain variable domain that has the sequence of SEQ ID NO:49.

In one embodiment, the recombinant protein includes a first antibody region having the sequence of SEQ ID NO:80 and a second antibody region including a light chain variable domain that has the sequence of SEQ ID NO:7 and a heavy chain variable domain that has the sequence of SEQ ID NO: 10.

In one embodiment, the recombinant protein includes a first antibody region having the sequence of SEQ ID NO:80 and a second antibody region including a light chain variable domain that has the sequence of SEQ ID NO:8 and a heavy chain variable domain that has the sequence of SEQ ID NO: 11.

In one embodiment, the recombinant protein includes a first antibody region having the sequence of SEQ ID NO:80 and a second antibody region including a light chain variable domain that has the sequence of SEQ ID NO:9 and a heavy chain variable domain that has the sequence of SEQ ID NO: 12.

In one embodiment, the recombinant protein includes a first antibody region having the sequence of SEQ ID NO:87 and a second antibody region including a light chain variable domain that has the sequence of SEQ ID NO:7 and a heavy chain variable domain that has the sequence of SEQ ID NO: 10.

In one embodiment, the recombinant protein includes a first antibody region having the sequence of SEQ ID NO:87 and a second antibody region including a light chain variable domain that has the sequence of SEQ ID NO:8 and a heavy chain variable domain that has the sequence of SEQ ID NO: 11.

In one embodiment, the recombinant protein includes a first antibody region having the sequence of SEQ ID NO:87 and a second antibody region including a light chain variable domain that has the sequence of SEQ ID NO:9 and a heavy chain variable domain that has the sequence of SEQ ID NO: 12.

In one embodiment, the recombinant protein includes a first antibody region having the sequence of SEQ ID NO:80 and a second antibody region including a light chain variable domain that has the sequence of SEQ ID NO:44 and a heavy chain variable domain that has the sequence of SEQ ID NO:47.

In one embodiment, the recombinant protein includes a first antibody region having the sequence of SEQ ID NO:80 and a second antibody region including a light chain variable domain that has the sequence of SEQ ID NO:45 and a heavy chain variable domain that has the sequence of SEQ ID NO:48.

In one embodiment, the recombinant protein includes a first antibody region having the sequence of SEQ ID NO:80 and a second antibody region including a light chain variable domain that has the sequence of SEQ ID NO:46 and a heavy chain variable domain that has the sequence of SEQ ID NO:49.

In one embodiment, the recombinant protein includes a first antibody region having the sequence of SEQ ID NO:87 and a second antibody region including a light chain variable domain that has the sequence of SEQ ID NO:44 and a heavy chain variable domain that has the sequence of SEQ ID NO:47.

In one embodiment, the recombinant protein includes a first antibody region having the sequence of SEQ ID NO:87 and a second antibody region including a light chain variable domain that has the sequence of SEQ ID NO:45 and a heavy chain variable domain that has the sequence of SEQ ID NO:48.

In one embodiment, the recombinant protein includes a first antibody region having the sequence of SEQ ID NO:87 and a second antibody region including a light chain variable domain that has the sequence of SEQ ID NO:46 and a heavy chain variable domain that has the sequence of SEQ ID NO:49.

### III. Pharmaceutical Compositions

Agents of the invention (e.g., recombinant proteins provided herein) are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

The compositions can be administered for therapeutic or prophylactic treatments. In therapeutic applications, compositions are administered to a patient suffering from a disease (e.g., cancer) in a “therapeutically effective dose.” Amounts effective for this use will depend upon the severity of the disease and the general state of the patient's health. Single or multiple administrations of the compositions may be administered depending on the dosage and frequency as required and tolerated by the patient. A “patient” or “subject” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administrations contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

In an aspect is provided a pharmaceutical composition including a therapeutically effective amount of a recombinant protein as provided herein including embodiments thereof and a pharmaceutically acceptable excipient.

A therapeutically effective amount as provided herein refers to an amount effective to achieve its intended purpose. The actual amount effective for a particular application will depend, inter alia, on the condition being treated. When administered in methods to treat a disease, the pharmaceutical compositions described herein will contain an amount of active recombinant protein (e.g., bispecific antibody) effective to achieve the desired result, e.g., modulating the activity of a target molecule (e.g., BAFF-R), activating an effector cell (e.g., T cell) and/or reducing, eliminating, or slowing the progression of disease symptoms (e.g., cancer, autoimmune disease). Determination of a therapeutically effective amount of a recombinant protein (e.g., bispecific antibody) provided herein is well within the capabilities of those skilled in the art, especially in light of the detailed disclosure herein.

### IV. Methods of Treatment

In an aspect is provided a method of treating cancer in a subject in need thereof, the method including administering to a subject a therapeutically effective amount of a recombinant protein (e.g., bispecific antibody) as described herein, including embodiments thereof, thereby treating cancer in the subject. In embodiments, the cancer is lymphoma, leukemia or myeloma. In embodiments, the cancer is lymphoma. In embodiments, the lymphoma is mantle cell lymphoma, follicular lymphoma, diffuse large B-cell lymphoma, marginal zone lymphoma or Burkitt's lymphoma. In embodiments, the lymphoma is mantle cell lymphoma. In embodiments, the lymphoma is follicular lymphoma. In embodiments, the lymphoma is diffuse large B-cell lymphoma. In embodiments, the lymphoma is marginal zone lymphoma. In embodiments, the lymphoma is Burkitt's lymphoma.

In embodiments, the cancer is leukemia. In embodiments, the leukemia is lymphoblastic leukemia, chronic lymphocytic leukemia or hairy cell leukemia. In embodiments, the leukemia is lymphoblastic leukemia. In embodiments, the leukemia is chronic lymphocytic leukemia. In embodiments, the leukemia is hairy cell leukemia.

In embodiments, the cancer is myeloma. In embodiments, the myeloma is multiple myeloma.

In embodiments, the method further includes administering to the subject a second therapeutic agent. In embodiments, the therapeutic agent is a chimeric monoclonal antibody capable of binding a CD20 antigen. In embodiments, the therapeutic agent is rituximab. The term “rituximab” refers in a customary sense to the monoclonal antibody against the protein CD20 identified by the ATC code L01XC02.

In another aspect, a method of treating an autoimmune disease in a subject in need thereof is provided. The method includes administering to the subject a therapeutically effective amount of a recombinant protein (e.g., bispecific antibody) as provided herein including embodiments thereof, thereby treating an autoimmune disease in the subject. In embodiments, the autoimmune disease is rheumatoid arthritis, systemic Lupus erythematosus, multiple sclerosis, glomerulonephritis, Sjogren's Syndrome or autoimmune hemolytic anemia. In embodiments, the autoimmune disease is rheumatoid arthritis. In embodiments, the autoimmune disease is systemic Lupus erythematosus. In embodiments, the autoimmune disease is multiple sclerosis. In embodiments, the autoimmune disease is glomerulonephritis. In embodiments, the autoimmune disease is Sjogren's Syndrome. In embodiments, the autoimmune disease is autoimmune hemolytic anemia. In embodiments, the method further includes administering to the subject a second therapeutic agent.

A therapeutically effective amount as provided herein refers to an amount effective to achieve its intended purpose. The actual amount effective for a particular application will depend, inter alia, on the condition being treated. When administered in methods to treat a disease, the pharmaceutical compositions described herein will contain an amount of active recombinant protein (BAFF-R bispecific antibody) provided herein including embodiments thereof, effective to achieve the desired result, e.g., modulating the activity of a target molecule (e.g., BAFF-R), activating an effector cell and/or reducing, eliminating, or slowing the progression of disease symptoms (e.g., cancer, autoimmune disease). Determination of a therapeutically effective amount of a recombinant protein (BAFF-R bispecific antibody) provided herein is well within the capabilities of those skilled in the art, especially in light of the detailed disclosure herein.

Acceptable carriers, excipients or stabilizers are nontoxic to recipients at the dosages and concentrations employed, and include buffers such as phosphate, citrate, or acetate at a pH typically of 5.0 to 8.0, optionally 6.0 to 7.0; salts such as sodium chloride, potassium chloride, and the like to make isotonic; antioxidants; preservatives; low molecular weight polypeptides; proteins; hydrophilic polymers such as polysorbate 80; amino acids such as glycine; carbohydrates; chelating agents; sugars; and other standard ingredients known to those skilled in the art {Remington: The Science and Practice of Pharmacy, 22nd Edition, Loyd V. Allen et al., editors, Pharmaceutical Press (2012)). The mAb can be present at a concentration of 0.1-100 mg/ml, e.g., 1-10 mg/ml or 10-50 mg/ml, for example 5, 10, 20, 30, 40, 50 or 60 mg/ml.

A pharmaceutical composition including a recombinant protein (BAFF-R bispecific antibody) provided herein including embodiments thereof, can be administered by a variety of methods known in the art. The route and/or mode of administration vary depending upon the desired results. Optionally, administration is intravenous, intramuscular, intraperitoneal, or subcutaneous, or administered proximal to the site of the target. Pharmaceutically acceptable excipients can be suitable for intravenous, intramuscular, subcutaneous, parenteral, spinal or epidermal administration (e.g., by injection or infusion).

Pharmaceutical compositions of the recombinant protein (BAFF-R bispecific antibody) provided herein including embodiments thereof can be prepared in accordance with methods well known and routinely practiced in the art. See, e.g., Remington: The Science and Practice of Pharmacy, 22nd Edition, Loyd V. Allen et al., editors, Pharmaceutical Press (2012); and Sustained and Controlled Release Drug Delivery Systems, J R. Robinson, ed., Marcel Dekker, Inc., New York, 1978. Pharmaceutical compositions are preferably manufactured under GMP conditions. Typically, a therapeutically effective dose or efficacious dose of the recombinant protein (BAFF-R bispecific antibody) provided herein including embodiments thereof, is employed in the pharmaceutical compositions. The recombinant protein (BAFF-R bispecific antibody) provided herein including embodiments thereof can be formulated into pharmaceutically acceptable dosage forms by conventional methods known to those of skill in the art. Dosage regimens are adjusted to provide the optimum desired response {e.g., a therapeutic response). For example, a single bolus may be administered, several divided doses may be administered over time or the dose may be proportionally reduced or increased as indicated by the exigencies of the therapeutic situation. It may be advantageous to formulate the recombinant protein (BAFF-R bispecific antibody) provided herein including embodiments thereof, in combination with other therapies or agents. It can be advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of the recombinant protein (BAFF-R bispecific antibody) provided herein including embodiments thereof, calculated to produce the desired therapeutic effect in association with the required pharmaceutical excipient.

Actual dosage levels of the active ingredients in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions employed, the route of administration, the time of administration, the rate of excretion of the particular antibody being employed, the duration of the treatment, other drugs, compounds and/or materials used in combination with the particular compositions employed, the age, sex, weight, condition, general health and prior medical history of the patient being treated, and like factors.

A physician or veterinarian can start doses of the recombinant proteins (BAFF-R bispecific antibody) provided herein including embodiments thereof employed in the pharmaceutical composition at levels lower than that required to achieve the desired therapeutic effect and gradually increase the dosage until the desired effect is achieved. In general, effective doses of the compositions may vary depending upon many different factors, including the specific disease or condition to be treated, means of administration, target site, physiological state of the patient, whether the patient is human or an animal, other medications administered, and whether treatment is prophylactic or therapeutic. Treatment dosages need to be titrated to optimize safety and efficacy. For administration with a bispecific antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two or three weeks or once a month or once every 3 to 6 months.

The recombinant protein (BAFF-R bispecific antibody) provided herein including embodiments thereof can be administered on multiple occasions. Intervals between single dosages can be weekly, monthly or yearly. Intervals can also be irregular as indicated by measuring blood levels of the bispecific antibody in the patient. In some methods, dosage is adjusted to achieve a plasma antibody concentration of 1-1000 μg/ml and in some methods 25-300 μg/ml.

Alternatively, the recombinant protein (BAFF-R bispecific antibody) provided herein including embodiments thereof can be administered as a sustained release formulation, in which case less frequent administration is required. Dosage and frequency vary depending on the half-life of the recombinant protein (BAFF-R bispecific antibody) provided herein including embodiments thereof in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### EXAMPLES

### Example

Bispecific T-cell engagers (BiTE) are a class of bispecific monoclonal antibodies that can be used as a targeted immunotherapeutic agent. Differing from traditional monoclonal antibodies, BiTE antibodies consist of two unique variable fragments arranged as two single-chain variable fragments (scFv). One scFv targets and engages T cells via the CD3 molecule and the second will target a cell surface antigen on tumor cells. Targeting the two molecules will not only link T cells to tumor cells but also activate the T cell via CD3 signaling. The activated T cell will then exert cytotoxic activity against tumor cells.

A BAFF-R BiTE (bi-specific antibody) was constructed with scFv derived from the heavy and light chains of a previously developed humanized anti-BAFF-R monoclonal antibody as disclosed in PCT/US2017/036178, supra. Preliminary testing of the BAFF-R BiTE has demonstrated strong specific binding to BAFF-R and human T cells. The BAFF-R BiTE is capable of eliciting a strong T-cell response against BAFF-R-expressing lymphoma and leukemia lines in vitro. Healthy donor B cells were also subject to cytotoxicity in the presence of BAFF-R BiTEs and T-cell effectors due to their normal BAFF-R expression.

A BAFF-R BiTE bi-specific antibody was generated by knob-hole technology. To facilitate heterodimer formation, the knobs-into-holes construct was applied to the bispecific antibody. Gels were run under reducing and non-reducing conditions for BAFF-R BiTE (H90Bi). Controls included control/irrelevant BiTE (Cont. Bi, 4-4Bi) and the original humanized BAFF-R antibody (H90 Ab also referred to herein as C90).

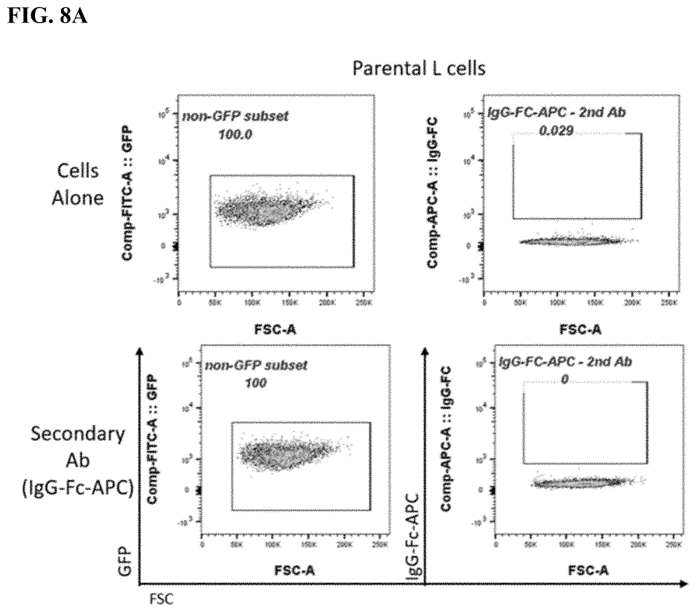

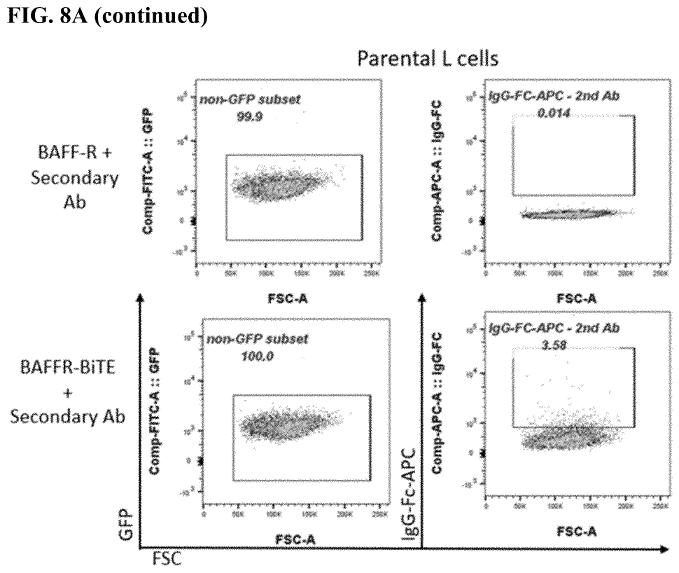

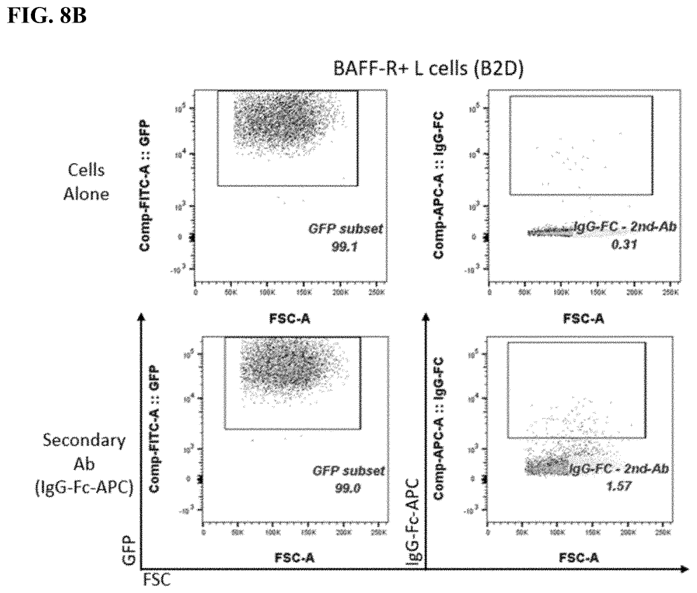

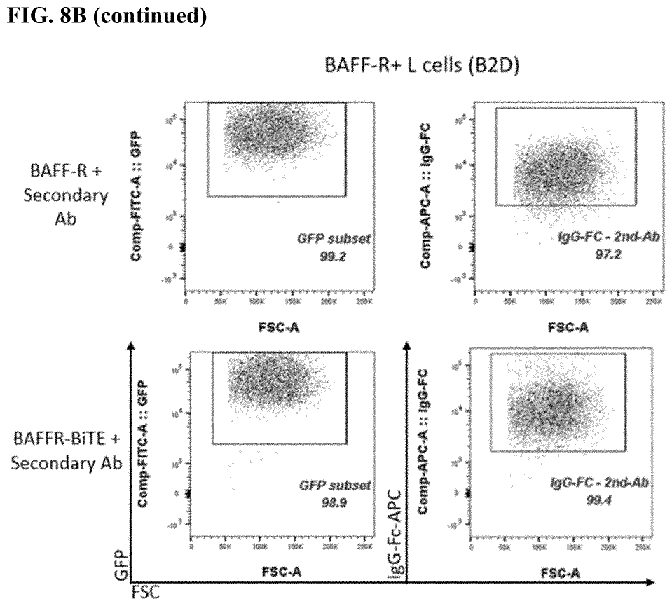

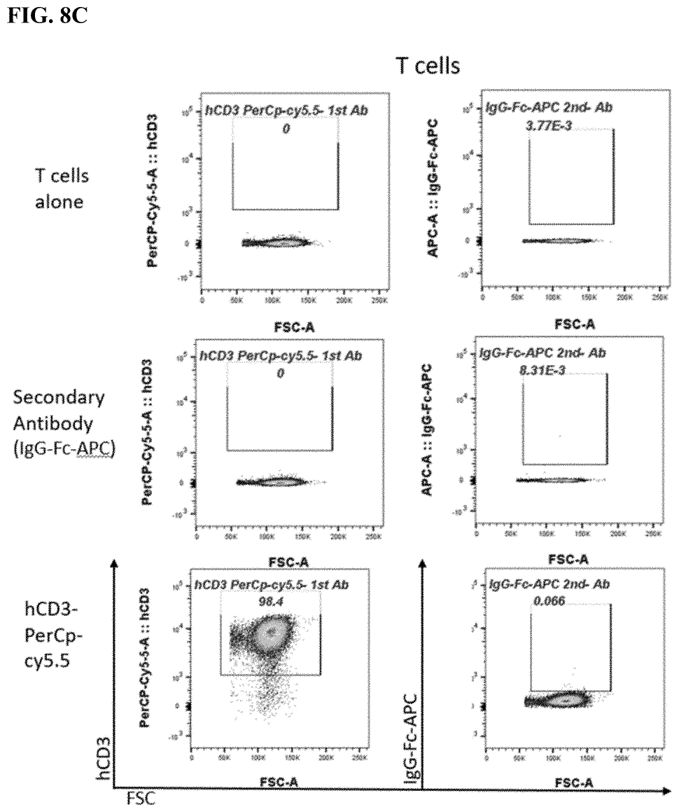

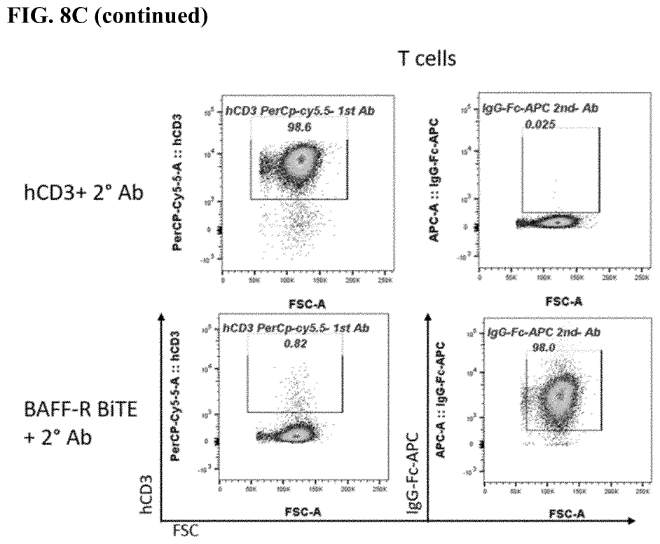

Binding specificity of bispecific antibodies was measured using FACS histograms of BAFF-R/CD3 bispecific antibody (H90 BiTE) binding wildtype mouse fibroblast L cells, engineered BAFF-R expressing L cells (B2D L cells), and T cells isolated from healthy donors as shown in FIG. 2. Antibody concentrations ranged from 0.05 μg to 5 μg. An irrelevant BiTE antibody was used as a control. Anti-human IgG antibody was used as the secondary antibody. BAFF-R BiTE binding was further evaluated using FACS plots and were confirmed to independently bind BAFF-R and T cells as shown in FIGS. 8A-8C.

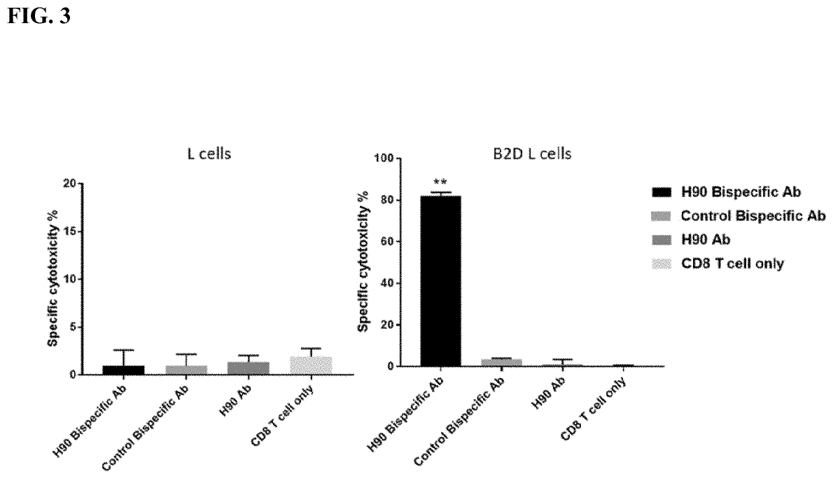

Specific cytotoxicity of bispecific antibodies was measured against BAFF-R-expressing L cells. Plots show calculated specific cytotoxicity percentage from a chromium release assay (FIG. 3). BAFF-R/CD3 bispecific antibody (H90 BiTE) was incubated with chromium labeled target wildtype or BAFF-R expressing L cells along with isolated effector CD8 T cells from a single healthy donor at an effector to target (E:T) ratio of 10:1 for 4 hours. Controls included control/irrelevant BiTE (Cont. Bi, 4-4Bi), the original humanized BAFF-R antibody (H90 Ab), or CD8 T cells only (no antibody).

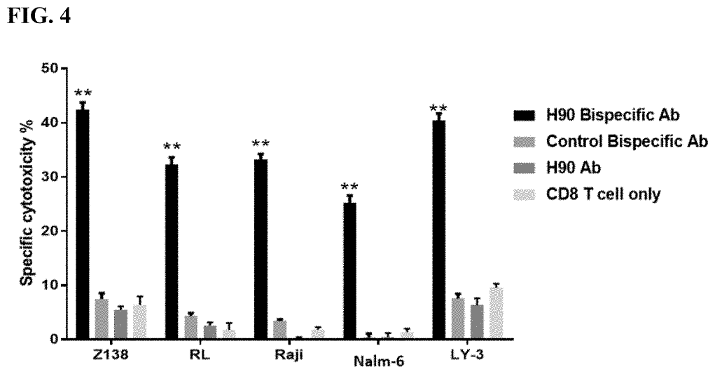

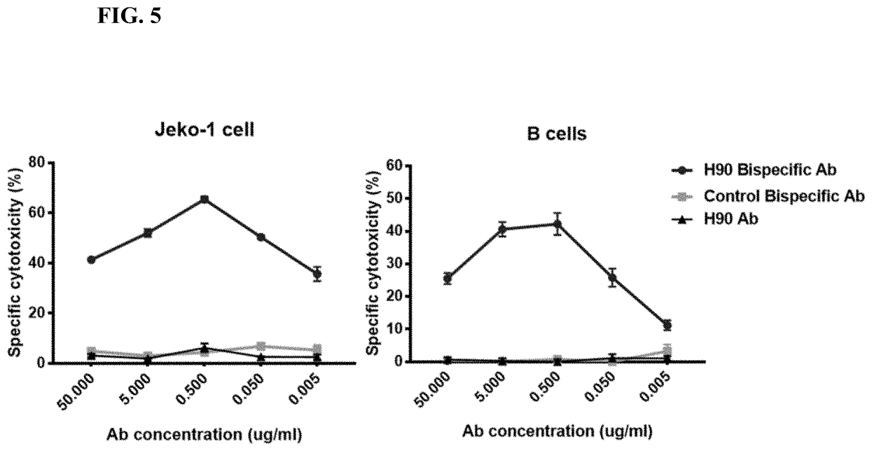

Cytotoxicity of bispecific antibodies was measured against malignant B-cell lines. Plots show calculated specific cytotoxicity percentage from a chromium release assay against various malignant B-cells lines as shown (FIG. 4). BAFF-R/CD3 bispecific antibody (H90 BITE) was incubated with chromium labeled target cell lines as shown along with isolated effector CD8 T cells from a single healthy donor at an effector to target (E:T) ratio of 10:1 for 4 hours. Controls included control/irrelevant BiTE (Cont. Bi, 4-4Bi), the original humanized BAFF-R antibody (H90 Ab), or CD8 T cells only (no antibody).

Dose-dependent cytotoxicity of bispecific antibodies was measured. Plots show calculated specific cytotoxicity percentage from a chromium release assay against JeKo-1 mantle cell lymphoma and normal isolated B cells (FIG. 5). Varying antibody concentrations of BAFF-R/CD3 bispecific antibody (H90 BiTE) was incubated with chromium labeled target JeKo-1 or normal B cells along with isolated effector CD8 T cells from a single healthy donor at an effector to target (E:T) ratio of 10:1 for 4 hours. Controls included control/irrelevant BiTE (Cont. Bi, 4-4Bi), the original humanized BAFF-R antibody (H90 Ab), or CD8 T cells only (no antibody).

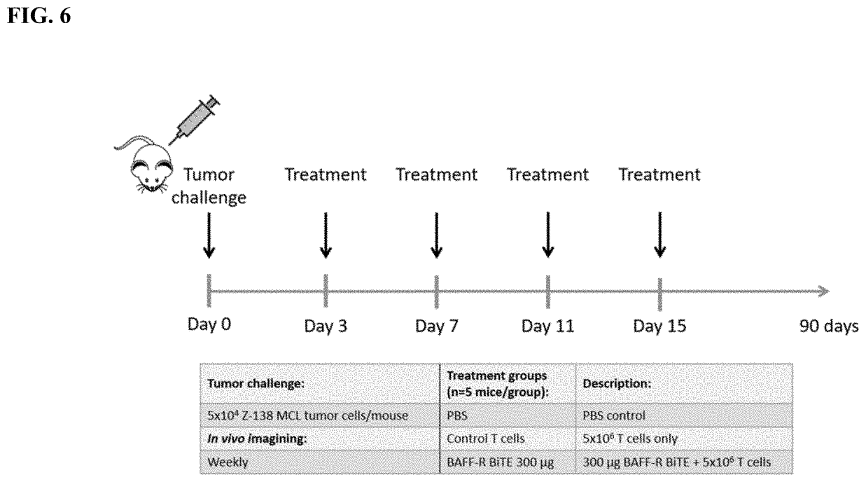

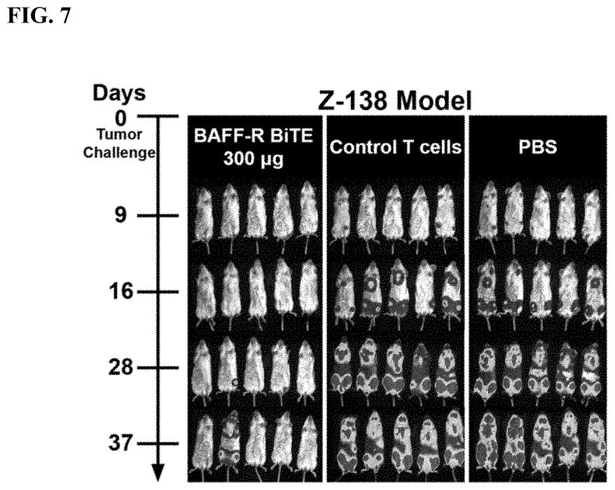

An exemplary treatment schedule using the Z-138 MCL tumor mouse model, where mice were treated with BAFF-R BiTE, is depicted in the schematic of FIG. 6. Mice were treated to evaluate the efficacy of the BiTE in reducing tumor growth. The effect of administration of BAFF-R BiTE, control T cells and PBS on tumor growth in these animals is shown in FIG. 7.

INFORMAL SEQUENCE LISTING

Humanized C90/H90 CDR L1: ESVDNYGIS (SEQ ID NO: 1)
Humanized C90/H90 CDR L2: AAS (SEQ ID NO: 2)
Humanized C90/H90 CDR L3: QQSKEVPWT (SEQ ID NO: 3)
Humanized C90/H90 CDR H1: GDSITSGY (SEQ ID NO: 4)
Humanized C90/H90 CDR H2: ISYSGST (SEQ ID NO: 5)
Humanized C90/H90 CDR H3: ASPNYPFYAMDY (SEQ ID NO:
6)
Humanized C90-1 Light Chain Variable domain protein
sequence
METDTLLLWVLLLWVPGSTGEIVLTQSPATLSLSPGERATLSCRASESVDN
YGISFLNWFQQKPGQAPRLLIYAASNRATGIPARFSGSGSGTDFTLTISSL
EPEDFAVYYCQQSKEVPWTFGGGTKVEIKRTV (SEQ ID NO: 7)
Humanized C90-2 Light Chain Variable domain protein
sequence
METDTLLLWVLLLWVPGSTGDIVLTQSPATLSLSPGERATLSCRASESVDN
YGISFMNWFQQKPGQAPRLLIYAASNRATGIPARFSGSGSGTDFTLTISSL
EPEDFAVYYCQQSKEVPWTFGGGTKVEIKRTV (SEQ ID NO: 8)
Humanized C90-3 Light Chain Variable domain
protein sequence
METDTLLLWVLLLWVPGSTGDIVMTQSPSSLSASVGDRVTITCRASESVDN
YGISFMNWFQQKPGKAPKLLIYAASNLGSGVPSRFSGSGSGTDFTLTISSL
QPEDFATYYCQQSKEVPWTFGQGTKVEIKRTV (SEQ ID NO: 9)
Humanized C90-1 Heavy Chain Variable domain
protein sequence
MDPKGSLSWRILLFLSLAFELSYGQVQLQESGPGLVKPSQTLSLTCTVSGD
SITSGYWNWIRQHPGKGLEYIGYISYSGSTYYNPSLKSRVTISRDTSKNQF
SLKLSSVTAADTAVYYCASPNYPFYAMDYWGQGTLVTVSS (SEQ ID
NO: 10)
Humanized C90-2 Heavy Chain Variable domain
protein sequence
MDPKGSLSWRILLFLSLAFELSYGEVQLQESGPGLVKPSQTLSLTCTVSGD
SITSGYWNWIRQHPGKGLEYIGYISYSGSTYYNPSLKSRVTISRDTSKNQY
SLKLSSVTAADTAVYYCASPNYPFYAMDYWGQGTLVTVSS (SEQ ID
NO: 11)
Humanized C90-3 Heavy Chain Variable domain
protein sequence
MDPKGSLSWRILLFLSLAFELSYGEVQLQESGPGLVKPSETLSLTCSVSGD
SITSGYWNWIRQPPGKGLEYIGYISYSGSTYYNPSLKSRVTISRDTSKNQY
SLRLSSVTAADTALYYCASPNYPFYAMDYWGQGTRVTVSS (SEQ ID
NO: 12)
Humanized C90-1 FR L1:
METDTLLLWVLLLWVPGSTGEIVLTQSPATLSLSPGERATLSCRAS (SEQ
ID NO: 13)
Humanized C90-1 FR L2:
FLNWFQQKPGQAPRLLIY (SEQ ID NO: 14)
Humanized C90-1 FR L3:
NRATGIPARFSGSGSGTDFTLTISSLEPEDFAVYYC (SEQ ID NO:
15)
Humanized C90-1 FR L4:
FGGGTKVEIKRTV (SEQ ID NO: 16)
Humanized C90-2 FR L1:
METDTLLLWVLLLWVPGSTGDIVLTQSPATLSLSPGERATLSCRAS
(SEQ ID NO: 17)
Humanized C90-2 FR L2:
FMNWFQQKPGQAPRLLIY (SEQ ID NO: 18)
Humanized C90-2 FR L3:
NRATGIPARFSGSGSGTDFTLTISSLEPEDFAVYYC (SEQ ID NO:
19)
Humanized C90-2 FR L4:
FGGGTKVEIKRTV (SEQ ID NO: 20)
Humanized C90-3 FR L1:
METDTLLLWVLLLWVPGSTGDIVMTQSPSSLSASVGDRVTITCRAS
(SEQ ID NO: 21)
Humanized C90-3 FR L2:
FMNWFQQKPGKAPKLLIY (SEQ ID NO: 22)
Humanized C90-3 FR L3:
NLGSGVPSRFSGSGSGTDFTLTISSLQPEDFATYYC (SEQ ID NO:
23)
Humanized C90-3 FR L4:
FGQGTKVEIKRTV (SEQ ID NO: 24)
Humanized C90-1 FR H1:
MDPKGSLSWRILLFLSLAFELSYGQVQLQESGPGLVKPSQTLSLTCTVS
(SEQ ID NO: 25)
Humanized C90-1 FR H2:
WNWIRQHPGKGLEYIGY (SEQ ID NO: 26)
Humanized C90-1 FR H3:
YYNPSLKSRVTISRDTSKNQFSLKLSSVTAADTAVYYC (SEQ ID NO:
27)
Humanized C90-1 FR H4:
WGQGTLVTVSS (SEQ ID NO: 28)
Humanized C90-2 FR H1:
MDPKGSLSWRILLFLSLAFELSYGEVQLQESGPGLVKPSQTLSLTCTVS
(SEQ ID NO: 29)
Humanized C90-2 FR H2:
WNWIRQHPGKGLEYIGY (SEQ ID NO: 30)
Humanized C90-2 FR H3:
YYNPSLKSRVTISRDTSKNQYSLKLSSVTAADTAVYYC (SEQ ID NO:
31)
Humanized C90-2 FR H4:
WGQGTLVTVSS (SEQ ID NO: 32)
Humanized C90-3 FR H1:
MDPKGSLSWRILLFLSLAFELSYGEVQLQESGPGLVKPSETLSLTCSVS
(SEQ ID NO: 33)
Humanized C90-3 FR H2:
WNWIRQPPGKGLEYIGY (SEQ ID NO: 34)
Humanized C90-3 FR H3:
YYNPSLKSRVTISRDTSKNQYSLRLSSVTAADTALYYC (SEQ ID NO:
35)
Humanized C90-3 FR H4: WGQGTRVTVSS (SEQ ID NO: 36)
Humanized C90 or C55 linker: GGGSGGGSGGGS (SEQ ID
NO: 37)
Humanized C55 CDR L1: QDISNY (SEQ ID NO: 38)
Humanized C55 CDR L2: YTS (SEQ ID NO: 39)
Humanized C55 CDR L3: HQFSELPWT (SEQ ID NO: 40)
Humanized C55 CDR H1: GFSLSTSGMG (SEQ ID NO: 41)
Humanized C55 CDR H2: IWWDDDK (SEQ ID NO: 42)
Humanized C55 CDR H3: ARSFGYGLDY (SEQ ID NO: 43)
Humanized C55-1 Light Chain Variable domain
protein sequence
METDTLLLWVLLLWVPGSTGDIQMTQSPSSLSASVGDRVTITCQASQDISN
YLNWYQQKPGKAPKLLIYYTSSLHTGVPSRFSGSGSGTDYTFTISSLQPED
IATYYCHQFSELPWTFGGGTKVEIKRTV (SEQ ID NO: 44)
Humanized C55-2 Light Chain Variable domain protein
sequence
METDTLLLWVLLLWVPGSTGDIQMTQSPSSLSASVGDRVTITCSASQDISN
YLNWYQQKPGKAPKLLIYYTSSLHTGVPSRFSGSGSGTDYTLTISSLQPED
IATYYCHQFSELPWTFGGGTKVEIKRTV (SEQ ID NO: 45)
Humanized C55-3 Light Chain Variable domain protein
sequence
METDTLLLWVLLLWVPGSTGDIQMTQSPSSLSASVGDRVTITCQASQDISN
YLNWYQQKPGKTPKLLIYYTSSLHTGVPSRFSGSGSGTDYTLTISSLQPED
IATYYCHQFSELPWTFGGGTKVEIKRTV (SEQ ID NO: 46)
Humanized C55-1 Heavy Chain Variable domain
protein sequence
MDPKGSLSWRILLFLSLAFELSYGQVTLKESGPTLVKPTQTLTLTCTFSGF
SLSTSGMGVGWIRQPPGKALEWLAHIWWDDDKYYNPSLKSRLTITKDTSKN
QVVLTMTNMDPVDTATYYCARSFGYGLDYWGQGTLVTVSS (SEQ ID
NO: 47)
Humanized C55-2 Heavy Chain Variable domain
protein sequence
MDPKGSLSWRILLFLSLAFELSYGQVTLKESGPTLVKPTQTLTLTCTFSGF
SLSTSGMGVGWIRQPPGKALEWLAHIWWDDDKYYNSSLKSRLTITKDTSKN
QVVLTMTNMDPVDTATYYCARSFGYGLDYWGQGTLVTVSS (SEQ ID
NO: 48)
Humanized C55-3 Heavy Chain Variable domain
protein sequence
MDPKGSLSWRILLFLSLAFELSYGQVTLKESGPALVKPTQTLTLTCTFSGF
SLSTSGMGVGWIRQPPGKALEWLAHIWWDDDKYYNTSLKSRLTITKDTSKN
QVVLKMTNMDPVDTATYYCARSFGYGLDYWGQGTLVTVSS (SEQ ID
NO: 49)
Humanized C55-1 FR L1:
METDTLLLWVLLLWVPGSTGDIQMTQSPSSLSASVGDRVTITCQAS
(SEQ ID NO: 50)
Humanized C55-1 FR L2:
LNWYQQKPGKAPKLLIY (SEQ ID NO: 51)
Humanized C55-1 FR L3:
SLHTGVPSRFSGSGSGTDYTFTISSLQPEDIATYYC (SEQ ID NO:
52)
Humanized C55-1 FR L4:
FGGGTKVEIKRTV (SEQ ID NO: 53)
Humanized C55-2 FR L1:
METDTLLLWVLLLWVPGSTGDIQMTQSPSSLSASVGDRVTITCSAS
(SEQ ID NO: 54)
Humanized C55-2 FR L2:
LNWYQQKPGKAPKLLIY (SEQ ID NO: 55)
Humanized C55-2 FR L3:
SLHTGVPSRFSGSGSGTDYTLTISSLQPEDIATYYC (SEQ ID NO:
56)
Humanized C55-2 FR L4:
FGGGTKVEIKRTV (SEQ ID NO: 57)
Humanized C55-3 FR L1:
METDTLLLWVLLLWVPGSTGDIQMTQSPSSLSASVGDRVTITCQAS
(SEQ ID NO: 58)
Humanized C55-3 FR L2:
LNWYQQKPGKTPKLLIY (SEQ ID NO: 59)
Humanized C55-3 FR L3:
SLHTGVPSRFSGSGSGTDYTLTISSLQPEDIATYYC (SEQ ID NO:
60)
Humanized C55-3 FR L4:
FGGGTKVEIKRTV (SEQ ID NO: 61)
Humanized C55-1 FR H1:
MDPKGSLSWRILLFLSLAFELSYGQVTLKESGPTLVKPTQTLTLTCTFS
(SEQ ID NO: 62)
Humanized C55-1 FR H2:
VGWIRQPPGKALEWLAH (SEQ ID NO: 63)
Humanized C55-1 FR H3:
YYNPSLKSRLTITKDTSKNQVVLTMTNMDPVDTATYYC (SEQ ID NO:
64)
Humanized C55-1 FR H4:
WGQGTLVTVSS (SEQ ID NO: 65)
Humanized C55-2 FR H1:
MDPKGSLSWRILLFLSLAFELSYGQVTLKESGPTLVKPTQTLTLTCTFS
(SEQ ID NO: 66)
Humanized C55-2 FR H2:
VGWIRQPPGKALEWLAH (SEQ ID NO: 67)
Humanized C55-2 FR H3:
YYNSSLKSRLTITKDTSKNQVVLTMTNMDPVDTATYYC (SEQ ID NO:
68)
Humanized C55-2 FR H4:
WGQGTLVTVSS (SEQ ID NO: 69)
Humanized C55-3 FR H1:
MDPKGSLSWRILLFLSLAFELSYGQVTLKESGPALVKPTQTLTLTCTFS
(SEQ ID NO: 70)
Humanized C55-3 FR H2:
VGWIRQPPGKALEWLAH (SEQ ID NO: 71)
Humanized C55-3 FR H3:
YYNTSLKSRLTITKDTSKNQVVLKMTNMDPVDTATYYC
(SEQ ID NO: 72)
Humanized C55-3 FR H4:
WGQGTLVTVSS (SEQ ID NO: 73)
Anti-CD3 scFv 1 CDR L1:
TGAVTSGNY (SEQ ID NO: 74)
Anti-CD3 scFv 1 CDR L2:
GTK (SEQ ID NO: 75)
Anti-CD3 scFv 1 CDR L3:
VLWYSNRWV (SEQ ID NO: 76)
Anti-CD3 scFv 1 CDR Hl:
GFTFNKYA (SEQ ID NO: 77)
Anti-CD3 scFv 1 CDR H2:
IRSKYNNYAT (SEQ ID NO: 78)
Anti-CD3 scFv 1 CDR H3:
VRHGNFGNSYISYWAY (SEQ ID NO: 79)
Anti-CD3 scFv 1 protein sequence
EVQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHG
NFGNSYISYWAYWGQGTLVTVSSGGGGSGGGGSGGGGSQTVVTQEPSLTVS
PGGTVTLTCGSSTGAVTSGNYPNWVQQKPGQAPRGLIGGTKFLAPGTPARF
SGSLLGGKAALTLSGVQPEDEAEYYCVLWYSNRWVFGGGTKLTVL
(SEQ ID NO: 80)
Anti-CD3 scFv 2 CDR Ll:
SSVSY (SEQ ID NO: 81)
Anti-CD3 scFv 2 CDR L2:
DTS (SEQ ID NO: 82)
Anti-CD3 scFv 2 CDR L3:
QQWSSNPFT (SEQ ID NO: 83)
Anti-CD3 scFv 2 CDR H1:
GYTFTRYT (SEQ ID NO: 84)
Anti-CD3 scFv 2 CDR H2:
INPSRGYT (SEQ ID NO: 85)
Anti-CD3 scFv 2 CDR H3:
ARYYDDHYCLDY (SEQ ID NO: 86)
Anti-CD3 scFv 2 protein sequence
QVQLVQSGGGVVQPGRSLRLSCKASGYTFTRYTMHWVRQAPGKGLEWIGYI
NPSRGYTNYNQKVKDRFTISRDNSKNTAFLQMDSLRPEDTGVYFCARYYDD
HYCLDYWGQGTPVTVSSGGGGSGGGGSGGGGSDIQMTQSPSSLSASVGDRV
TITCSASSSVSYMNWYQQTPGKAPKRWIYDTSKLASGVPSRFSGSGSGTDY
TFTISSLQPEDIATYYCQQWSSNPFTFGQGTKLQITR (SEQ ID NO:
87)

### EMBODIMENTS

### Embodiment 1

A recombinant protein comprising:
(i) a first antibody region capable of binding an effector cell ligand; and
(ii) a second antibody region, comprising:
(a) a light chain variable domain comprising a CDR L1 as set forth in SEQ ID NO:1, a CDR L2 as set forth in SEQ ID NO:2 and a CDR L3 as set forth in SEQ ID NO:3; and (b) a heavy chain variable domain comprising a CDR H1 as set forth in SEQ ID NO:4, a CDR H2 as set forth in SEQ ID NO:5, and a CDR H3 as set forth in SEQ ID NO:6.

### Embodiment 2

The recombinant protein of Embodiment 1, wherein said light chain variable domain comprises the sequence of SEQ ID NO:7.

### Embodiment 3

The recombinant protein of Embodiment 1 or 2, wherein said heavy chain variable domain comprises the sequence of SEQ ID NO: 10.

### Embodiment 4

The recombinant protein of any one of Embodiments 1-3, wherein said light chain variable domain comprises a FR L1 as set forth in SEQ ID NO: 13, a FR L2 as set forth in SEQ ID NO: 14, FR L3 as set forth in SEQ ID NO: 15 and a FR L4 as set forth in SEQ ID NO:16.

### Embodiment 5

The recombinant protein of any one of Embodiments 1-4, wherein said heavy chain variable domain comprises a FR H1 as set forth in SEQ ID NO:25, a FR H2 as set forth in SEQ ID NO:26, FR H3 as set forth in SEQ ID NO:27 and a FR H4 as set forth in SEQ ID NO:28.

### Embodiment 6

The recombinant protein of Embodiment 1, wherein said light chain variable domain comprises the sequence of SEQ ID NO: 8.

### Embodiment 7

The recombinant protein of Embodiment 1 or 6, wherein said heavy chain variable domain comprises the sequence of SEQ ID NO: 11.

### Embodiment 8

The recombinant protein of any one of Embodiments 1, 6, or 7, wherein said light chain variable domain comprises a FR L1 as set forth in SEQ ID NO:17, a FR L2 as set forth in SEQ ID NO:18, FR L3 as set forth in SEQ ID NO:19 and a FR L4 as set forth in SEQ ID NO:20.

### Embodiment 9

The recombinant protein of any one of Embodiments 1 or 6-8, wherein said heavy chain variable domain comprises a FR H1 as set forth in SEQ ID NO:29, a FR H2 as set forth in SEQ ID NO:30, FR H3 as set forth in SEQ ID NO:31 and a FR H4 as set forth in SEQ ID NO:32.

### Embodiment 10

The recombinant protein of Embodiment 1, wherein said light chain variable domain comprises the sequence of SEQ ID NO:9.

### Embodiment 11

The recombinant protein of Embodiment 1 or 10, wherein said heavy chain variable domain comprises the sequence of SEQ ID NO: 12.

### Embodiment 12

The recombinant protein of any one of Embodiments 1, 10, or 11, wherein said light chain variable domain comprises a FR L1 as set forth in SEQ ID NO:21, a FR L2 as set forth in SEQ ID NO:22, FR L3 as set forth in SEQ ID NO:23 and a FR L4 as set forth in SEQ ID NO:24.

### Embodiment 13

The recombinant protein of any one of Embodiments 1 or 10-12, wherein said heavy chain variable domain comprises a FR H1 as set forth in SEQ ID NO:33, a FR H2 as set forth in SEQ ID NO:34, FR H3 as set forth in SEQ ID NO:35 and a FR H4 as set forth in SEQ ID NO:36.

### Embodiment 14

A recombinant protein comprising:
(i) a first antibody region capable of binding an effector cell ligand; and
(ii) a second antibody region, comprising:
(a) a light chain variable domain comprising a CDR L1 as set forth in SEQ ID NO:38, a CDR L2 as set forth in SEQ ID NO:39 and a CDR L3 as set forth in SEQ ID NO:40; and
(b) a heavy chain variable domain comprising a CDR H1 as set forth in SEQ ID NO:41, a CDR H2 as set forth in SEQ ID NO:42, and a CDR H3 as set forth in SEQ ID NO:44.

### Embodiment 15

The recombinant protein of Embodiment 14, wherein said light chain variable domain comprises the sequence of SEQ ID NO:44.

### Embodiment 16

The recombinant protein of Embodiment 14 or 15, wherein said heavy chain variable domain comprises the sequence of SEQ ID NO:47.

### Embodiment 17

The recombinant protein of any one of Embodiments 14-16, wherein said light chain variable domain comprises a FR L1 as set forth in SEQ ID NO:50, a FR L2 as set forth in SEQ ID NO:51, FR L3 as set forth in SEQ ID NO:52 and a FR L4 as set forth in SEQ ID NO:53.

### Embodiment 18

The recombinant protein of any one of Embodiments 14-17, wherein said heavy chain variable domain comprises a FR H1 as set forth in SEQ ID NO:62, a FR H2 as set forth in SEQ ID NO:63, FR H3 as set forth in SEQ ID NO:64 and a FR H4 as set forth in SEQ ID NO:65.

### Embodiment 19

The recombinant protein of Embodiment 14, wherein said light chain variable domain comprises the sequence of SEQ ID NO:45.

### Embodiment 20

The recombinant protein of Embodiment 14 or 19, wherein said heavy chain variable domain comprises the sequence of SEQ ID NO:48.

### Embodiment 21

The recombinant protein of any one of Embodiments 14, 19 or 20, wherein said light chain variable domain comprises a FR L1 as set forth in SEQ ID NO:54, a FR L2 as set forth in SEQ ID NO:55, FR L3 as set forth in SEQ ID NO:56 and a FR L4 as set forth in SEQ ID NO:57.

### Embodiment 22

The recombinant protein of any one of Embodiments 14 or 19-21, wherein said heavy chain variable domain comprises a FR H1 as set forth in SEQ ID NO:66, a FR H2 as set forth in SEQ ID NO:67, FR H3 as set forth in SEQ ID NO:68 and a FR H4 as set forth in SEQ ID NO:69.

### Embodiment 23

The recombinant protein of Embodiment 14, wherein said light chain variable domain comprises the sequence of SEQ ID NO:46.

### Embodiment 24

The recombinant protein of Embodiment 14 or 23 14 or 23, wherein said heavy chain variable domain comprises the sequence of SEQ ID NO:49.

### Embodiment 25

The recombinant protein of any one of Embodiments 14, 23 or 24, wherein said light chain variable domain comprises a FR L1 as set forth in SEQ ID NO:58, a FR L2 as set forth in SEQ ID NO:59, FR L3 as set forth in SEQ ID NO:60 and a FR L4 as set forth in SEQ ID NO:61.

### Embodiment 26

The recombinant protein of any one of Embodiments 14 or 23-25, wherein said heavy chain variable domain comprises a FR H1 as set forth in SEQ ID NO:70, a FR H2 as set forth in SEQ ID NO:71, FR H3 as set forth in SEQ ID NO:72 and a FR H4 as set forth in SEQ ID NO:73.

### Embodiment 27

The recombinant protein of any one of Embodiments 1-26, wherein said first antibody region is a first Fab′ fragment or said second antibody region is a second Fab′ fragment.

### Embodiment 28

The recombinant protein of any one of Embodiments 1-26, wherein said first antibody region is a first single chain variable fragment (scFv) or said second antibody region is a second single chain variable fragment (scFv).

### Embodiment 29

The recombinant protein of any one of Embodiments 1-28, wherein said second antibody region is capable of binding a BAFF-R protein with an equilibrium dissociation constant (K_{D}) of less than about 5 nM.

### Embodiment 30

The recombinant protein of any one of Embodiments 1-29, wherein said second antibody region is capable of binding a BAFF-R protein with an equilibrium dissociation constant (K_{D}) of less than about 4 nM.

### Embodiment 31

The recombinant protein of any one of Embodiments 1-30, wherein said first antibody region comprises:
(a) a light chain variable domain comprising a CDR L1 as set forth in SEQ ID NO:74, a CDR L2 as set forth in SEQ ID NO:75 and a CDR L3 as set forth in SEQ ID NO:76; and
(b) a heavy chain variable domain comprising a CDR H1 as set forth in SEQ ID NO:77, a CDR H2 as set forth in SEQ ID NO:78, and a CDR H3 as set forth in SEQ ID NO:79.

### Embodiment 32

The recombinant protein of any one of Embodiments 1-30, wherein said first antibody region comprises:
(a) a light chain variable domain comprising a CDR L1 as set forth in SEQ ID NO:81, a CDR L2 as set forth in SEQ ID NO:82 and a CDR L3 as set forth in SEQ ID NO:83; and
(b) a heavy chain variable domain comprising a CDR H1 as set forth in SEQ ID NO:84 a CDR H2 as set forth in SEQ ID NO:85, and a CDR H3 as set forth in SEQ ID NO:86.

### Embodiment 33

The recombinant protein of any one of Embodiments 1-32, wherein said first antibody region is a first single chain variable fragment (scFv).

### Embodiment 34

The recombinant protein of Embodiment 33, wherein said first scFv comprises the sequence of SEQ ID NO:80 or SEQ ID NO:87.

### Embodiment 35

The recombinant protein of any one of Embodiments 1-34, wherein said effector cell ligand is a CD3 protein.

### Embodiment 36

The recombinant protein of any one of Embodiment 1-35, wherein said second antibody region is bound to a BAFF-R protein.

### Embodiment 37

The recombinant protein of Embodiment 36, wherein said BAFF-R protein is a human BAFF-R protein.

### Embodiment 38

The recombinant protein of Embodiment 36, wherein said BAFF-R protein forms part of a cell.

### Embodiment 39

The recombinant protein of Embodiment 38, wherein said BAFF-R protein is expressed on the surface of said cell.

### Embodiment 40

The recombinant protein of Embodiment 39, wherein said cell is a lymphoid cell.

### Embodiment 41

The recombinant protein of Embodiment 39, wherein said cell is a B cell.

### Embodiment 42

The recombinant protein of Embodiment 39, wherein said cell is a cancer cell.

### Embodiment 43

The recombinant protein of Embodiment 42, wherein said cancer cell is a lymphoma cell.

### Embodiment 44

A pharmaceutical composition comprising a therapeutically effective amount of a recombinant protein of any one of Embodiments 1-43 and a pharmaceutically acceptable excipient.

### Embodiment 45

A method of treating cancer in a subject in need thereof, said method comprising administering to a subject a therapeutically effective amount of a recombinant protein of any one of Embodiments 1-43, thereby treating cancer in said subject.

### Embodiment 46

The method of Embodiment 45, wherein said cancer is lymphoma, leukemia or myeloma.

### Embodiment 47

A method of treating an autoimmune disease in a subject in need thereof, said method comprising administering to a subject a therapeutically effective amount of a recombinant protein of any one of Embodiments 1-43, thereby treating an autoimmune disease in said subject.

What is claimed is:

1. A recombinant protein comprising:
(i) a first antibody region capable of binding an effector cell ligand; and
(ii) a second antibody region, comprising:
(a) a light chain variable domain comprising a CDR L1 as set forth in SEQ ID NO:1, a CDR L2 as set forth in SEQ ID NO:2 and a CDR L3 as set forth in SEQ ID NO:3; and
(b) a heavy chain variable domain comprising a CDR H1 as set forth in SEQ ID NO:4, a CDR H2 as set forth in SEQ ID NO:5, and a CDR H3 as set forth in SEQ ID NO:6.

2. The recombinant protein of claim 1, wherein said light chain variable domain comprises the sequence of SEQ ID NO:7.

3. The recombinant protein of claim 1, wherein said heavy chain variable domain comprises the sequence of SEQ ID NO:10.

4. The recombinant protein of claim 1, wherein said light chain variable domain comprises a FR L1 as set forth in SEQ ID NO:13, a FR L2 as set forth in SEQ ID NO:14, FR L3 as set forth in SEQ ID NO:15 and a FR L4 as set forth in SEQ ID NO:16.

5. The recombinant protein of claim 1, wherein said heavy chain variable domain comprises a FR H1 as set forth in SEQ ID NO:25, a FR H2 as set forth in SEQ ID NO:26, FR H3 as set forth in SEQ ID NO:27 and a FR H4 as set forth in SEQ ID NO:28.

6. The recombinant protein of claim 1, wherein said light chain variable domain comprises the sequence of SEQ ID NO:8.

7. The recombinant protein of claim 1, wherein said heavy chain variable domain comprises the sequence of SEQ ID NO:11.

8. The recombinant protein of claim 1, wherein said light chain variable domain comprises a FR L1 as set forth in SEQ ID NO:17, a FR L2 as set forth in SEQ ID NO:18, FR L3 as set forth in SEQ ID NO:19 and a FR L4 as set forth in SEQ ID NO:20.

9. The recombinant protein of claim 1, wherein said heavy chain variable domain comprises a FR H1 as set forth in SEQ ID NO:29, a FR H2 as set forth in SEQ ID NO:30, FR H3 as set forth in SEQ ID NO:31 and a FR H4 as set forth in SEQ ID NO:32.

10. The recombinant protein of claim 1, wherein said light chain variable domain comprises the sequence of SEQ ID NO:9.

11. The recombinant protein of claim 1, wherein said heavy chain variable domain comprises the sequence of SEQ ID NO: 12.

12. The recombinant protein of claim 1, wherein said light chain variable domain comprises a FR L1 as set forth in SEQ ID NO:21, a FR L2 as set forth in SEQ ID NO:22, FR L3 as set forth in SEQ ID NO:23 and a FR L4 as set forth in SEQ ID NO:24.

13. The recombinant protein of claim 1, wherein said heavy chain variable domain comprises a FR H1 as set forth in SEQ ID NO:33, a FR H2 as set forth in SEQ ID NO:34, FR H3 as set forth in SEQ ID NO:35 and a FR H4 as set forth in SEQ ID NO:36.

14. A recombinant protein comprising:
(i) a first antibody region capable of binding an effector cell ligand; and
(ii) a second antibody region, comprising:
(a) a light chain variable domain comprising a CDR L1 as set forth in SEQ ID NO:38, a CDR L2 as set forth in SEQ ID NO:39 and a CDR L3 as set forth in SEQ ID NO:40; and
(b) a heavy chain variable domain comprising a CDR H1 as set forth in SEQ ID NO:41, a CDR H2 as set forth in SEQ ID NO:42, and a CDR H3 as set forth in SEQ ID NO:43.

15. The recombinant protein of claim 14, wherein said light chain variable domain comprises the sequence of SEQ ID NO:44.

16. The recombinant protein of claim 14, wherein said heavy chain variable domain comprises the sequence of SEQ ID NO:47.

17. The recombinant protein of claim 14, wherein said light chain variable domain comprises a FR L1 as set forth in SEQ ID NO:50, a FR L2 as set forth in SEQ ID NO:51, FR L3 as set forth in SEQ ID NO:52 and a FR L4 as set forth in SEQ ID NO:53.

18. The recombinant protein of claim 14, wherein said heavy chain variable domain comprises a FR H1 as set forth in SEQ ID NO:62, a FR H2 as set forth in SEQ ID NO:63, FR H3 as set forth in SEQ ID NO:64 and a FR H4 as set forth in SEQ ID NO:65.

19. The recombinant protein of claim 14, wherein said light chain variable domain comprises the sequence of SEQ ID NO:45.

20. The recombinant protein of claim 14, wherein said heavy chain variable domain comprises the sequence of SEQ ID NO:48.

21. The recombinant protein of claim 14, wherein said light chain variable domain comprises a FR L1 as set forth in SEQ ID NO:54, a FR L2 as set forth in SEQ ID NO:55, FR L3 as set forth in SEQ ID NO:56 and a FR L4 as set forth in SEQ ID NO:57.

22. The recombinant protein of claim 14, wherein said heavy chain variable domain comprises a FR H1 as set forth in SEQ ID NO:66, a FR H2 as set forth in SEQ ID NO:67, FR H3 as set forth in SEQ ID NO:68 and a FR H4 as set forth in SEQ ID NO:69.

23. The recombinant protein of claim 14, wherein said light chain variable domain comprises the sequence of SEQ ID NO:46.

24. The recombinant protein of claim 14, wherein said heavy chain variable domain comprises the sequence of SEQ ID NO:49.

25. The recombinant protein of claim 14, wherein said light chain variable domain comprises a FR L1 as set forth in SEQ ID NO:58, a FR L2 as set forth in SEQ ID NO:59, FR L3 as set forth in SEQ ID NO:60 and a FR L4 as set forth in SEQ ID NO:61.

26. The recombinant protein of any one of claim 14, wherein said heavy chain variable domain comprises a FR H1 as set forth in SEQ ID NO:70, a FR H2 as set forth in SEQ ID NO:71, FR H3 as set forth in SEQ ID NO:72 and a FR H4 as set forth in SEQ ID NO:73.

27. The recombinant protein of claim 1, wherein said first antibody region is a first Fab′ fragment or said second antibody region is a second Fab′ fragment.

28. The recombinant protein of claim 1, wherein said first antibody region is a first single chain variable fragment (scFv) or said second antibody region is a second single chain variable fragment (scFv).

29. The recombinant protein of claim 1, wherein said second antibody region is capable of binding a BAFF-R protein with an equilibrium dissociation constant (K_{D}) of less than about 5 nM.

30. The recombinant protein of claim 1, wherein said second antibody region is capable of binding a BAFF-R protein with an equilibrium dissociation constant (K_{D}) of less than about 4 nM.

31. The recombinant protein of claim 1, wherein said first antibody region comprises:
(a) a light chain variable domain comprising a CDR L1 as set forth in SEQ ID NO:74, a CDR L2 as set forth in SEQ ID NO:75 and a CDR L3 as set forth in SEQ ID NO:76; and
(b) a heavy chain variable domain comprising a CDR H1 as set forth in SEQ ID NO:77, a CDR H2 as set forth in SEQ ID NO:78, and a CDR H3 as set forth in SEQ ID NO:79.

32. The recombinant protein of claim 1, wherein said first antibody region comprises:
(a) a light chain variable domain comprising a CDR L1 as set forth in SEQ ID NO:81, a CDR L2 as set forth in SEQ ID NO:82 and a CDR L3 as set forth in SEQ ID NO:83; and
(b) a heavy chain variable domain comprising a CDR H1 as set forth in SEQ ID NO:84 a CDR H2 as set forth in SEQ ID NO:85, and a CDR H3 as set forth in SEQ ID NO:86.

33. The recombinant protein of claim 1, wherein said first antibody region is a first single chain variable fragment (scFv).

34. The recombinant protein of claim 33, wherein said first scFv comprises the sequence of SEQ ID NO: 80 or SEQ ID NO: 87.

35. The recombinant protein of claim 1, wherein said effector cell ligand is a CD3 protein.

36. The recombinant protein of claim 1, wherein said second antibody region is bound to a BAFF-R protein.

37. The recombinant protein of claim 36, wherein said BAFF-R protein is a human BAFF-R protein.

38. The recombinant protein of claim 36, wherein said BAFF-R protein forms part of a cell.

39. The recombinant protein of claim 38, wherein said BAFF-R protein is expressed on the surface of said cell.

40. The recombinant protein of claim 39, wherein said cell is a lymphoid cell.

41. The recombinant protein of claim 39, wherein said cell is a B cell.

42. The recombinant protein of claim 39, wherein said cell is a cancer cell.

43. The recombinant protein of claim 42, wherein said cancer cell is a lymphoma cell.

44. A pharmaceutical composition comprising a therapeutically effective amount of a recombinant protein of claim 1 and a pharmaceutically acceptable excipient.

45. A method of treating cancer in a subject in need thereof, said method comprising administering to a subject a therapeutically effective amount of a recombinant protein of claim 1, thereby treating cancer in said subject.

46. The method of claim 45, wherein said cancer is lymphoma, leukemia or myeloma.

47. A method of treating an autoimmune disease in a subject in need thereof, said method comprising administering to a subject a therapeutically effective amount of a recombinant protein of claim 1, thereby treating an autoimmune disease in said subject.
